(19) United States
(12) Patent Application Publication
(10) Pub. No.: US 2009/0191174 A1
(43) Pub. Date: Jul. 30, 2009

(54) BLOOD PROCESSING DEVICE AND ASSOCIATED SYSTEMS AND METHODS

(75) Inventors: Keith Boudreau, Beverly, MA (US); Dan Morgan, Salem, MA (US); Mike Haley, Marlboro, MA (US); Will Garcia De Quevedo, Miami, FL (US)

Correspondence Address: FOLEY & LARDNER LLP, 111 HUNTINGTON AVENUE, 26TH FLOOR, BOSTON, MA, 02199-7610, US

(73) Assignee: Zymequest, Inc.

(21) Appl. No.: 11/995,421

(22) PCT Filed: Jul. 26, 2006
(86) PCT No.: PCT/US06/28876

§ 371 Date: Dec. 12, 2008

Related U.S. Application Data
(60) Provisional application No. 60/703,021, filed on Jul. 26, 2005.

(51) Int. Cl. A61K 35/12; C12M 1/00; C12N 5/00; A61P 37/00

(52) U.S. Cl. 424/93.73; 435/283.1; 435/289.1; 435/375; 435/325

(57) ABSTRACT

The present invention is directed to an environmentally closed cell processing device, for the aseptic processing of blood cells. The device includes a continuous flow centrifuge, fluid reservoirs and fluid handling systems. Blood cells are processed by the present device, to remove their immunodominant antigens. Seroconverted cells and methods of treating subjects with these cells are also described.

### FIELD OF THE INVENTION

The following invention is directed to various aspects of a blood processing system, having a continuous flow centrifuge system and a fluid management system, the system being suitable for independent aseptic processing of multiple separate quantities of blood and blood cells.

### BACKGROUND

There are more than thirty blood group (or type) systems, one of the most important of which is the ABO system. This system is based on the presence or absence of antigens A and/or B. These antigens are found on the surface of erythrocytes and platelets as well as on the surface of endothelial and most epithelial cells. The major blood product used for transfusion is erythrocytes, which are red blood cells containing hemoglobin, the principal function of which is the transport of oxygen. Blood of group A contains antigen A on its erythrocytes. Similarly, blood of group B contains antigen B on its erythrocytes. Blood of group AB contains both antigens, and blood of group 0 contains neither antigen.

The blood group structures are glycoproteins or glycolipids and considerable work has been done to identify the specific structures making up the A and B determinants or antigens. The ABH blood group specificity is determined by the nature and linkage of monosaccharides at the ends of the carbohydrate chains. The carbohydrate chains are attached to a peptide (glycoprotein) or lipid (glycosphingolipid) backbone, which are attached to the cell membrane of the cells. The immunodominant monosaccharide determining type A specificity is a terminal alpha1-3 linked N-acetylgalactosamine (GalNAc), while the corresponding monosaccharide of B type specificity is an alpha1-3 linked galactose (Gal). Type O cells lack either of these monosaccharides at the termini of oligosaccharide chains, which instead are terminated with alpha1-2 linked fucose (Fuc) residues.

A great diversity of blood group ABH carbohydrate structures are found due to structural variations in the oligosaccharide chains that carry ABH immunodominant saccharides. Our pending U.S. patent application Ser. No. 11/049,271 lists structures reported in man and those that have been found on human red cells or in blood extracts. For a review, see, Clausen & Hakomori, Vox Sang 56(1): 1-20,1989). Red cells contain ABH antigens on N-linked glycoproteins and glycosphingolipids, while it is generally believed that O-linked glycans on erythrocytes glycoproteins, mainly glycophorins, are terminated by sialic acid and not with ABH antigens. Type I chain glycosphingolipids are not endogenous products of red cells, but rather adsorbed from plasma.

Blood group A and B exist in several subtypes. Blood group A subtypes are the most frequent, and there are three recognized major sub-types of blood type A. These sub-types are known as A1, A intermediate (Aint) and A2. There are both quantitative and qualitative differences that distinguish these three sub-types. Quantitatively, A1 erythrocytes have more antigenic A sites, i.e., terminal N-acetylgalactosamine residues, than Aint erythrocytes which in turn have more antigenic A sites than A2 erythrocytes. Qualitatively, A1 erythrocytes have a dual repeated A structure on a subset of glycosphingolipids, while A2 cells have an H structure on an internal A structure on a similar subset of glycolipids (Clausen et al., Proc. Natl. Acad. Sci. USA 82(4): 1199-203, 1985, Clausen et al., J. Biol. Chem. 261(3): 1380-7, 1986). These differences between A1 and weak A subtypes are thought to relate to differences in the kinetic properties of blood group A isoenzyme variants responsible for the formation of A antigens (Clausen et al., J. Biol. Chem. 261(3): 1388-92,1986). The differences of group B subtypes are believed to be solely of quantitative nature.

Blood of group A contains antibodies to antigen B. Conversely, blood of group B contains antibodies to antigen A. Blood of group AB has neither antibody, and blood group O has both. Antibodies to these and other carbohydrate defined blood group antigens are believed to be elicited by continuous exposure to microbial organism carrying related carbohydrate structures. An individual whose blood contains either (or both) of the anti-A or anti-B antibodies cannot receive a transfusion of blood containing the corresponding incompatible antigen(s). If an individual receives a transfusion of blood of an incompatible group, the blood transfusion recipient's antibodies coat the red blood cells of the transfused incompatible group and cause the transfused red blood cells to agglutinate, or stick together. Transfusion reactions and/or hemolysis (the destruction of red blood cells) may result therefrom.

In order to avoid red blood cell agglutination, transfusion reactions, and hemolysis, transfusion blood type is cross-matched against the blood type of the transfusion recipient. For example, a blood type A recipient can be safely transfused with type A blood, which contains compatible antigens. Because type O blood contains no A or B antigens, it can be transfused into any recipient with any blood type, i.e., recipients with blood types A, B, AB or O. Thus, type O blood is considered “universal”, and maybe used for all transfusions. Hence, it is desirable for blood banks to maintain large quantities of type O blood. However, there is a paucity of blood type O donors. Therefore, it is desirable and useful to remove the is immunodominant A and B antigens on types A, B and AB blood in order to maintain large quantities of universal blood products.

Hospitals require a constant blood supply for transfusion. After donors provide blood, regional blood centers are responsible for ABO typing, infectious disease testing, component manufacturing, and distribution of red blood cells to hospitals. The hospitals again, test the A, B, AB, O blood group and cross match the available blood units to the appropriate patients. Since group O blood can be transfused universally, there is a high demand for group O blood, in general, and especially in emergency situations where the delay caused by typing and matching is not acceptable. Furthermore, the processed blood has a relatively short shelf life of 42 days, after which it may not be transfused. The balancing of the inventory of red blood cells is extraordinarily complex. On a daily basis, the regional blood centers must match the demand for different blood groups with the available supply held at the blood centers, and at its hospital customer sites around the country. The individual blood units are constantly moved within the system in order to match daily variation in supply and demand. In fact, individual units are frequently moved three to four times within the system before finally being transfused. Even with the best efforts by the participants to ensure that each collected unit is ultimately transfused, 4% to 8% of all collected units outdate before transfusion and must be discarded. A processing system that would reproducibly convert A, B or AB type blood to O type blood would satisfy a crucial need in this field. The availability of O blood cells would improve red cell availability, substantially eliminate red cell outdating caused by the inability to match units with recipients within the 42 day outdate window, eliminate the need for the frequent reshipment of blood units in order to match the daily supply and demand, and eliminate the need for retesting for the blood type.

In an attempt to increase the supply of type O blood, methods have been developed for converting certain type A, B and AB blood to type O blood. Conversion of B cells to type O cells has been accomplished in the past. However, conversion of the more abundant A cells has only been achieved with the less abundant weak A subgroup cells. The major obstacle for development and utilization of enzyme converted universal O cells to date includes the cost and activity profiles of some enzymes, and inadequate commercial processing systems to supply universal type O cells. There remains a need in the art for improved blood processing systems.

### SUMMARY OF THE INVENTION

The present invention is directed to a device for the processing of cells, particularly blood cells, and more particularly erythrocytes. The device includes a continuous flow centrifuge, having a structural frame and having a plurality of spaced apart structural discs and a plurality of support tubes, the centrifuge further including a rotor, the rotor having bearings on each end and a drive system on one end, and the rotor having a hub further including a port on one end and a plurality of channels therethrough each channel terminating in an aperture through the hub wall surface; a plurality of chambers disposed along the axis of the rotor, the chambers including substantially flexible processing bags and substantially flexible expressor bags; the processing bags having ports aligning with one or more of the channel apertures on the rotor hub, and the expressor bags having ports aligning with one or more of the channel apertures on the rotor hub; a supply module having a fluid management cassette, the fluid management cassette further comprising a network of pathways and valves, the supply module being further connected to a plurality of fluid and/or air sources, and where the valves are adapted to a system control module; a multi-lumen tube connected at one end to the supply module and at the other end to the hub on the centrifuge rotor, the tube supported by support tube rollers and a rigid arm geared to the centrifuge drive system; a plurality of supply reservoirs in communication with the supply module; and a system control module in electrical communication with the valves, and the centrifuge drive system, the control module having a processor, an instruction set, a fault hub system, and user defined input controls. In one embodiment, the drive system uses a direct drive motor. In another embodiment, the centrifuge rotor is adapted a drive pulley and a motor distal to the rotor. In yet another embodiment, the valves include protruding valve seats circumscribed by flow-through wells. In still another embodiment, one or more of the supply reservoirs contain a quantity of a glycan modifying enzyme in a buffered solution. In even yet another embodiment, the glycan modifying enzyme is an alpha-N-acetylgalactosaminidase or an alpha-galactosidase, particularly one having substantial bioactivity at or about a neutral pH. In various embodiments, the buffered solution has a pH of 5.0 to 8.0, preferably a pH of 6.0 to 7.8, more preferably a pH of 6.5 to 7.5, even more preferably a pH of 6.8 to 7.5 and most preferably a pH of 7.0 to 7.3.

In another aspect, the invention provides a method of modifying blood cells comprising: introducing a preparation of isolated blood cells into the processing chambers of the device of claim 1, and reacting said isolated blood cells with a buffered solution of an alpha-N-acetylgalactosaminidase enzyme or an alpha-galactosidase enzyme, or both enzymes, thereby removing immunodominant antigens from the blood cells, separating the antigentically modified blood cells from the enzyme, buffer solution, and enzymatically cleaved antigen fragments by centrifugation and wash steps, and recovering the isolated blood cells. In certain embodiments, the isolated modified cells are then mixed with sterile blood storage buffers such as buffered saline or plasma (preferably type O), to an appropriate hematocrit, and are frozen or stored for eventual transfusion to a subject (human or animal). In various embodiments, the buffered solution has a pH of 5.0 to 8.0, preferably a pH of 6.0 to 7.8, more preferably a pH of pH 6.5 to 7.5, even more preferably a pH of 6.8 to 7.5 and most preferably a pH of 7.0 to 7.3.

In another aspect, the invention provides for a seroconverted blood cell, wherein the immunodominant antigens are removed by the cell modification method described. In one embodiment, the modified blood cell is not a serotype A, B, or AB cell, as determined by standard blood typing methods. In yet another aspect, the invention provides for a population of packed seroconverted blood cells, prepared using the device and methods described herein.

In another aspect, the invention provides for a method of treating a subject in need of blood. The subject may be a mammal, and preferably a human, having any of the common blood serotypes A, B or AB, and even O. The subject is provided with a quantity of the seroconverted cells, i.e., processed to remove the immunodominant antigens from the cells designed to be transfused. The transfused cells restore blood to the subject, thereby treating the disorder.

These and other features will be apparent to those of skill in the art, and without limiting the foregoing, the invention is thus described in terms of the claims appended below.

### DESCRIPTION OF THE FIGURES

FIGS. 1A and 1B are exploded views of the bag set seal and door.

FIGS. 2A-2F are schematic diagrams of a centrifuge component of the blood processing device.

FIG. 3 is an illustration of a fluid management cassette, which includes a plurality of flow-through valves that control fluid pathway communications.

FIG. 4 is a schematic drawing directed to a centrifuge processing bag.

FIG. 5A-5C are schematic drawings directed to various modifications of the processing bag design, designed to improve the flow of materials (enzymes, buffers, fluids and blood cells), into and out of the bags.

FIG. 6 is a schematic drawing directed to a multi-lumen tube.

FIGS. 7A and 7B represent an electrical block diagram, and host logic, respectively, for the system.

FIG. 8 is a diagram of the host software architecture.

FIGS. 9A, 9B and 9C illustrate the fault hub system.

### DETAILED DESCRIPTION

The present invention provides a blood processing system, having a continuous flow centrifuge system and a fluid management system, the system being suitable for independent aseptic processing of multiple separate quantities of blood and blood cells. The system provides an environmentally closed reaction and processing apparatus, that in a presently preferred embodiment, is used with various enzymes (e.g., glycan modifying polypeptides) and buffers described in our pending U.S. patent application Ser. No. 11/049,271, to modify the immunodominant antigens of blood cells. Nevertheless, while blood processing is the currently preferred application, and the system is referred to in that capacity, it can be used with a wide variety of fluids and substrates, not simply blood, for example polypeptide processing and digestion, polynucleotide processing and digestion, incubation of microorganisms including recombinant technologies. Thus, the system has broad application for uses requiring a self-contained and automated centrifuge and fluid processing system. Such applications will be evident from a description of the various elements of the invention, as set forth more fully below.

### Continuous Flow Centrifuge

Generally, cell processing requires steps in which cells or cell elements are separate from a liquid phase. This separation is typically accomplished by centrifugation. The present invention thus includes a centrifuge apparatus and more particularly, a centrifuge which works in conjunction with a cassette, rotor or other device having fluid retentive chambers and fluid flow tubing fixedly attached to the axis of the device.

In the context of mechanisms which have come to be known as continuous flow centrifuges, when a length of tubing is fixedly attached to the rotation axis of a device which contains the fluid material to be centrifuged, the entire length of tubing must be rotated by use of rotary seals or some other means to avoid twisting the tubing. A well known method for avoiding the use of rotary seals is to curve the length of tubing outwardly from the axis and around the outer edge of the circumference of the rotor, cassettes or the like and to rotate the tubing in an orbital fashion around the rotor/cassette at one-half times the rotational speed of the rotor/cassette itself. Such a method for eliminating tube twisting and apparati therefore are disclosed, for example, in U.S. Pat. Nos. 4,216,770, 4,419,089 and 4,389,206.

Problems inherent in such prior apparatuses which orbit the fluid flow tubing around the axis of centrifuge rotation are that the axis of rotation is disposed vertically, the tubing is routed through an axial shaft and the apparatus is driven by driving an axial shaft which requires a high aspect ratio and an elongated shaft which limit the rotational speed, render the apparatus instable and limits the ability of the user to mount a second cassette, rotor or the like on opposing sides of the chuck component of the apparatus.

In accordance with the foregoing, reference is also made to U.S. Pat. No. 5,665,048 that provides a centrifuge for rotating a fluid retentive housing having fluid input and output tubing fixedly connected to a rotation axis of the fluid retentive housing, the centrifuge comprising: a frame; a first rotatable mechanism having a rotation axis, the fluid retentive housing being coaxially mounted thereon for co-rotation therewith; a second rotatable mechanism having a rotation axis, the first and second rotation mechanism being coaxially mounted on the frame; the second rotatable mechanism having an outer circumferential surface engaged with a drive mechanism, the drive mechanism driving the outer circumferential surface such that the second rotatable mechanism rotates at a selected rotational speed X; the first rotatable mechanism being interconnected to the second rotatable mechanism such that the first rotatable mechanism rotates simultaneously with the second rotatable mechanism at a rotational speed of 2X.

The second rotatable mechanism includes a seat for holding a distal length of the output tubing which extends from the axis of the fluid retentive housing, wherein the distal length of the output tubing held by the seat is rotated around the rotation axis at the same rotational speed as the second rotatable mechanism. One of the problems associated with such an arrangement is that there is continuous friction between the tubing and the seat.

Therefore, in accordance with the present invention, there is provided an improvement in a centrifuge, and an improvement relating to fluid tubing by the support thereof. In accordance with the present invention, there is provided a centrifuge for rotating a fluid retentive housing such that one or more selected materials suspended in a fluid retained within the housing centrifuged upon rotation of the housing. The centrifuge includes a first rotatable mechanism having a rotation access with the fluid retentive housing being coaxially mounted on the first rotatable mechanism for co-rotation therewith. There is also provided a second rotatable mechanism having a rotation axis with the first and second rotatable mechanisms being coaxially interconnected for co-rotation around a common axis. Fluid tubing connected to the axis of the fluid retentive housing has a distal length that extends axially outwardly from the fluid retentive housing. In accordance with one embodiment of the present invention, the improvement comprises a support arm mounted to the second rotatable mechanism, a support tube for receiving there through at least part of the distal length of the fluid tubing, and a bearing member for rotatably supporting the support tube in said support arm whereby upon rotation of the first and second rotation mechanisms, the fluid tubing is free to either rotate with or rotate relative to the support tube so as to minimize friction between the fluid tubing and the support therefor.

In accordance with another embodiment of the present invention, there is provided a multi-lumen “skip ” rope comprising a plurality of elongated tubes for delivering one or more fluids between a first fluid containing mechanism and a fluid receiving rotatably driven rotor. One end of the rope is attached to the center of the driven rotor and the other end of the rope is attached to the first fluid retaining mechanism. The first fluid retaining mechanism is mounted on an opposing side of the rotor such that the point of attachment of the other end of the rope is substantially coaxial with an axis of the rotor. The aforementioned elongated tubes may comprise at least one tube disposed of in a spiral wrap. This may be either a single strand or a multi-strand wrap and may be either in a counterclockwise or clockwise direction. And also, in a single strand or a multi-strand, at one end the spiral wrap may be clockwise while at the other end counterclockwise and also optionally have a straight section there between. The skip rope fluid delivery system is described in greater detail below.

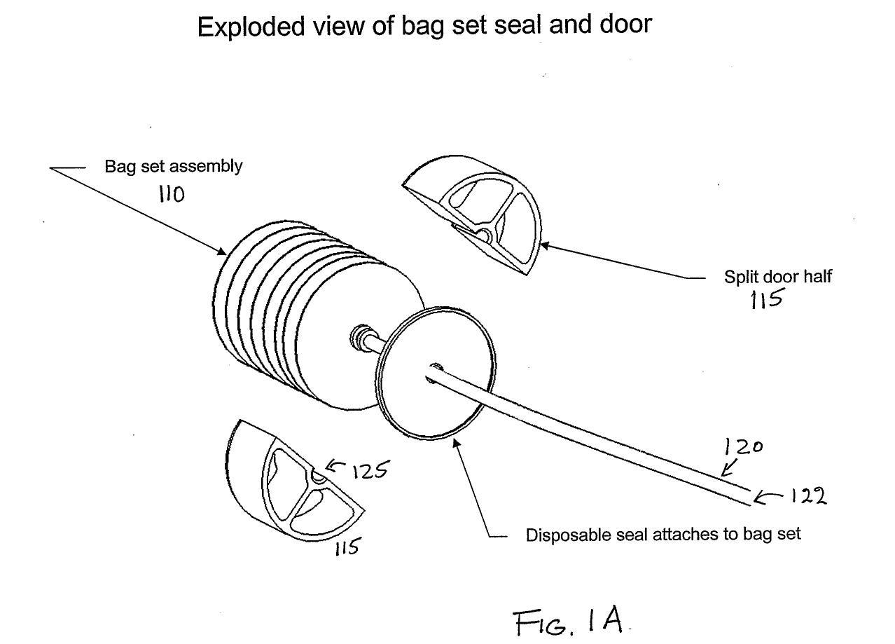

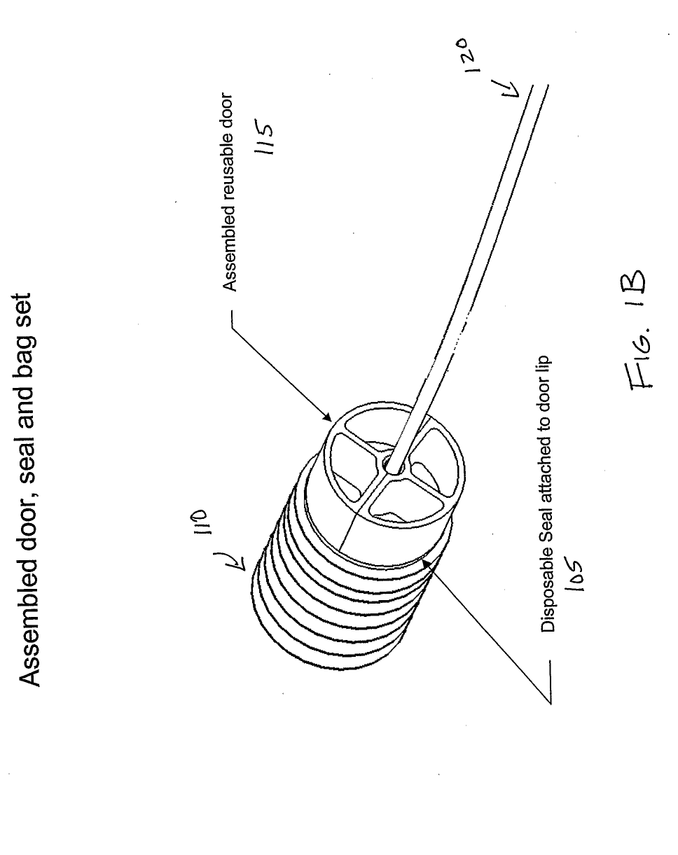

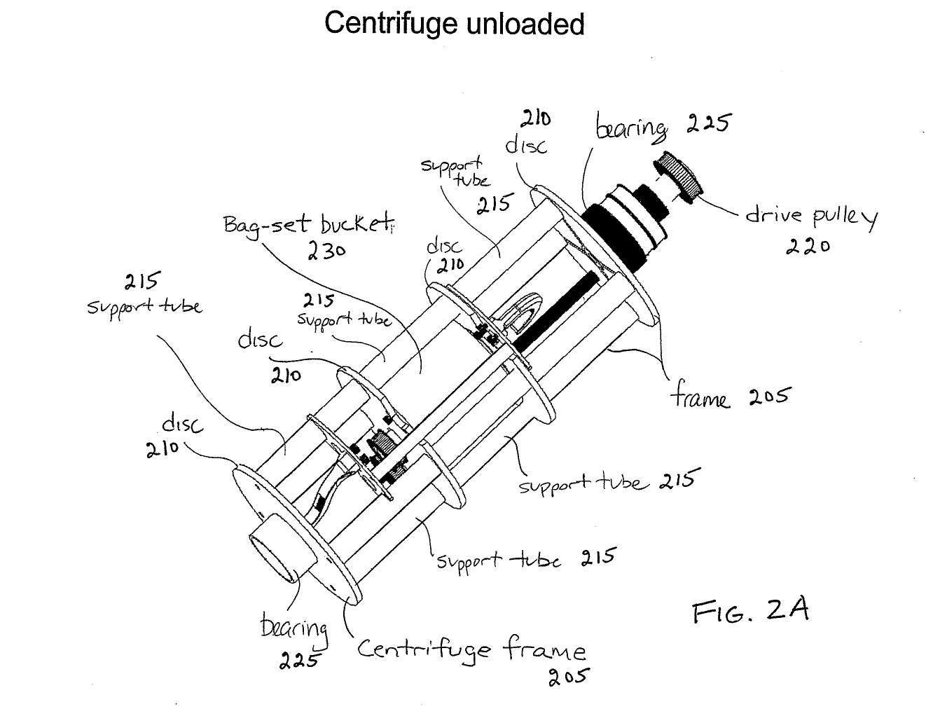

FIGS. 1A-1B illustrate an exploded view of a bag set assembly with disposable seal for attachment to a split door (expanded view with halves of split door shown apart. Suitable bags and bag sets 110 are described below. The disposable seal 105 engages a bag set on one side and on the opposite side contacts one side of the split door 115. The seal 105 also includes a center opening through which passes a multi-lumen skip rope 120, containing a plurality of lumens 122, each one being adapted to one or more bags of the bag set, i.e., in fluid communication or to a source of sterile air (see, our U.S. Pat. No. 7,008,366). The split door may also include a relief 125 for the multi-lumen rope. FIG. 1B illustrates the assembly of FIG. 1A with the split door in the closed position.

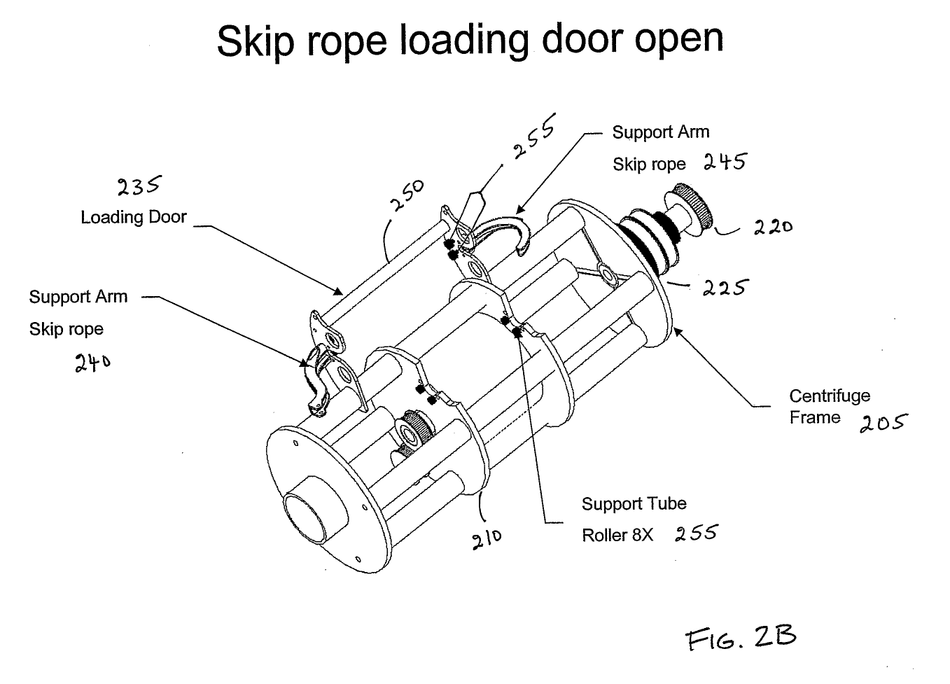

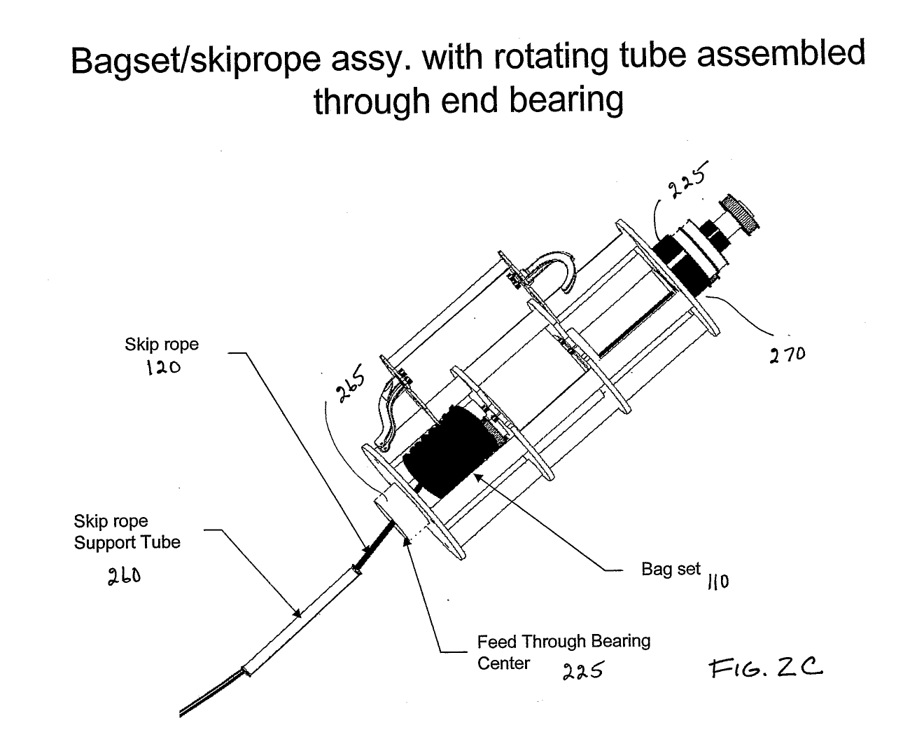

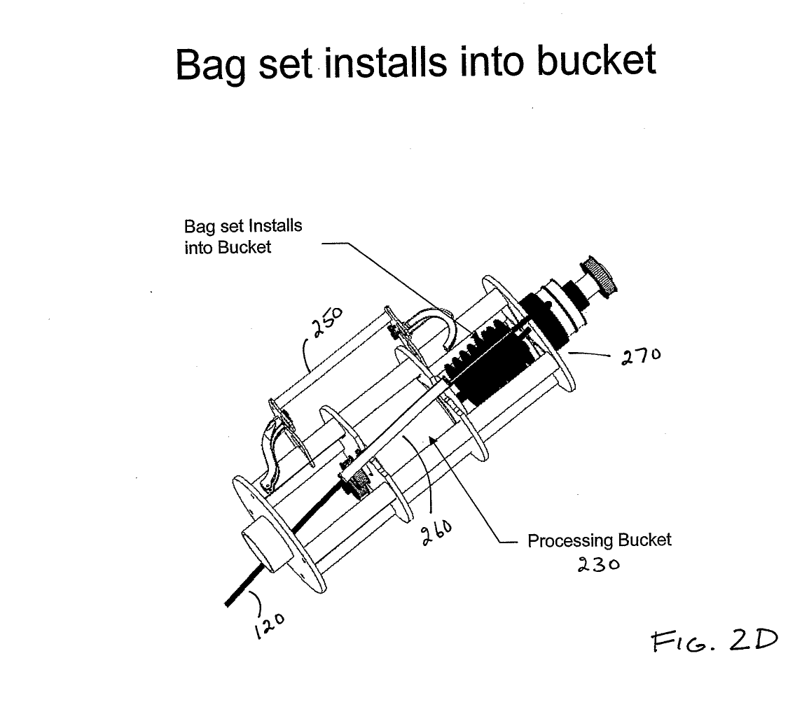

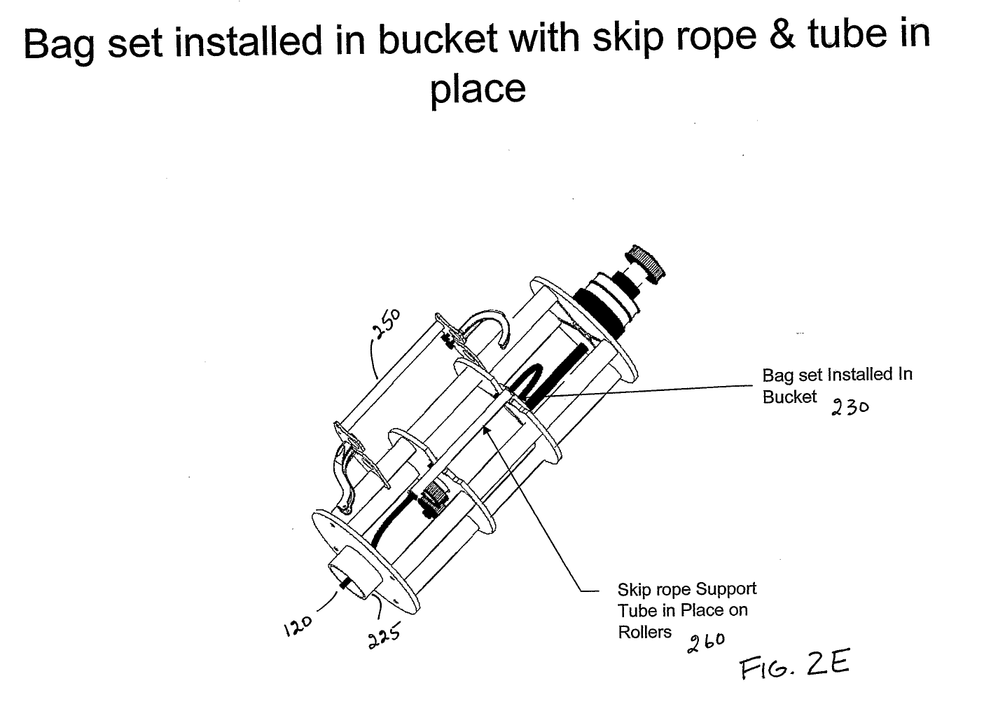

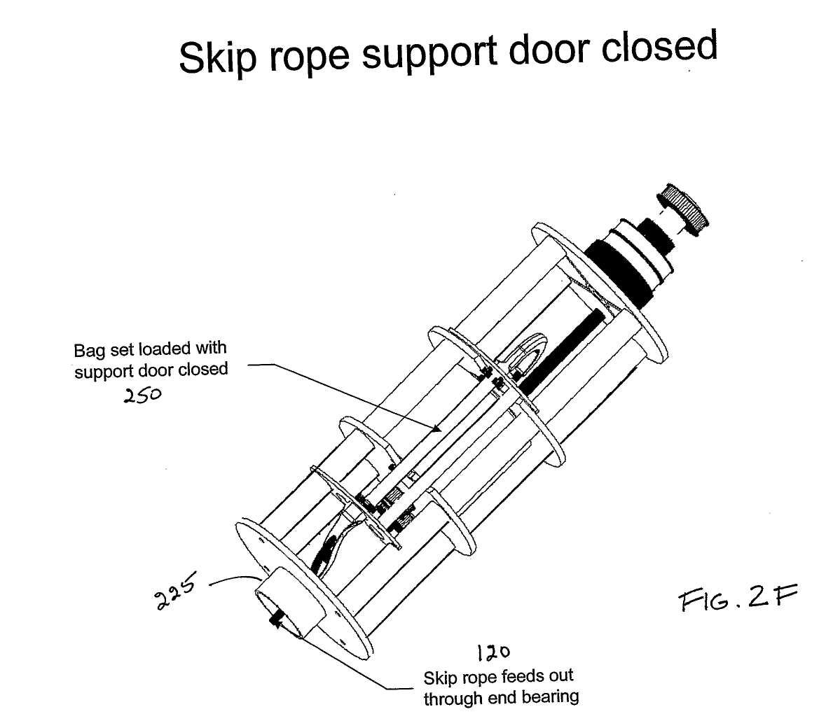

FIGS. 2A-2F are schematic diagrams of a centrifuge component of the blood processing device according to one of the embodiments of the present invention. As shown, the centrifuge includes a frame 205 having a plurality of spaced apart structural discs 210, and a plurality of support tubes 215. In this embodiment, the centrifuge also includes a drive pulley 220 provided for at an end, which receives a drive belt from a motor (not shown), and a bearing 225 on either end. In the center of the centrifuge is provided a bag-set bucket 230, which is designed to house one or more flexible processing chambers (and may also include expressor chambers).

A number of devices have been developed which incorporate a means of expressing (i.e., promoting the exit) fluid which has been removed from harvested cells. Disclosures relating to expression include U.S. Pat. No. 4,332,351 by Kellogg and Druger, U.S. Pat. No. 4,010,894 by Kellogg and Kruger, U.S. Pat. No. 4,007,871 by Jones and Kellogg, U.S. Pat. No. 3,737,097 by Jones et al., EP 00265795/EP B1 by Polaschegg, U.S. Pat. No. 4,934,995 by Cullis, U.S. Pat. No. 4,223,672 by Terman et al., and U.S. Pat. No. 4,213,561 by Bayham. Particularly preferred examples of flexible processing chambers suitable for use herein include those described in our United States Patent Application 20050143244. The apparatus thus generally can be described as including a centrifuge rotor having a round centrifuge chamber of selected volume, the centrifuge rotor being controllably rotatable around a central axis by a motor mechanism; a round expandable enclosure disposed within the centrifuge chamber having a rotation axis coincident with the central rotation axis and a flexible wall, the fluid container having a rotation axis and being coaxially receivable within the centrifuge chamber, the expandable enclosure being sealably connected to a source of an expresser fluid which has a density selected to be greater than the density of each of the selected one or more fluid materials disposed in the fluid container; a pump for controllably pumping a selected volume of the expresser fluid into and out of the expandable enclosure wherein the fluid container is receivable within the centrifuge chamber; a retaining mechanism for holding the fluid container within the centrifuge chamber in a coaxial position wherein the flexible wall of the fluid container is in contact with the flexible wall of the expandable enclosure.

The expandable enclosure preferably comprises a flexible membrane sealably attached to a surface of the rotor such that the centrifuge chamber is divided into a first chamber for receiving the fluid container and a second fluid sealed chamber for receiving the expresser fluid. The flexible wall of the expandable enclosure typically comprises an elastomeric sheet material. The apparatus further typically includes a heater or cooling mechanism having a control mechanism for selectively controlling the temperature of the expresser fluid.

Due to its higher density, the expresser fluid which is pumped into the expandable enclosure travels to a circumferential position within the expandable enclosure which is more radially outward from the central axis than a circumferential position to which the one or more selected fluid materials in the fluid container travel when the rotor is drivably rotated around the central axis.

The fluid container typically has a first radius and the second fluid sealed chamber typically has a second radius which is at least equal to the first radius of the fluid container, wherein the expresser fluid which is pumped into the second fluid sealed chamber travels to an outermost circumferential position within the second fluid sealed chamber which is more radially outward from the central axis than a circumferential position to which the one or more selected fluid materials in the fluid container travel when the rotor is drivably rotated around the central axis.

As shown in FIG. 2B, a skip rope loading door 235 is shown in an open position. The skip rope loading door includes a first end support arm 240 and a second end support arm 245, as well as a support tube 250 positioned therebetween for the skip rope. Also included, are four pairs of support tube rollers 255, which allow the support tube to rotate during centrifuging relative to rotation of the entire device.

FIG. 2C illustrates the bag-set/skip-rope assembly, with rotating support tube 260 assembled through the end bearing center. As shown in FIG. 2D, the bag-set 110 maybe inserted through the center of the end bearing 225, along with the skip-rope assembly. As shown, the bag-set is inserted through end 265 (FIG. 2C), then up and over the bag-set bucket 230, then into the bag-set bucket from the opposite end 270 (FIG. 2D). The skip-rope assembly and support tube 260 are then positioned in their appropriate positions adjacent the support tube rollers (of course, while the loading door is in an open position) (see FIG. 2E). The loading door is then closed (FIG. 2F), and an end of the skip-rope protrudes through the bearing center opening.

### Fluid Management System

The invention provides a cell processing system that can adjust the processing algorithm based on the type of the processed cells or the amount of the cells. Furthermore, it provides an automated interactive cell processing system that can assure uniform and reproducible processing condition of the same type of cells regardless of their amount being processed or the processing location. These objectives are accomplished in part by an automated fluid management system, accomplished by a processor that reads and executes instruction sets from software or permanently programmed instruction sets such as those on a programmable chip, the instruction sets being described in more detail below.

The system permits for distributing fluids from different sources to different destinations. The device that accomplishes this receives fluids from a plurality of different sources and distributes the fluids out of one or more ports to a destination. The device also receives fluid from the destination and transfers the fluid to one or more other ports, out to other destinations. The device includes a plurality of ports for receiving a plurality of fluids. The device includes a channel coupled to the plurality of ports, and a first port coupled to said channel. The first port is adapted to transfer fluid from said plurality of ports a first destination, and to receive fluid from said first destination. The device also includes a second port coupled to said channel adapted to transfer fluid received on said first port from said first destination to a second destination. A connector is provided that includes a first port to receive a first source of fluid, a second port to receive a second source of fluid, a first outlet in communication with the first port, and a second outlet in communication with the second port. The first and second outlets are adapted to be attached to first and second input ports of a device for distributing the first and second fluids to a particular destination. Chambers are provided for storing fluids that includes a first compartment for storing a first fluid, and a second compartment for storing a second fluid. Further examples of preferred fluid management systems include our U.S. Pat. No. 6,425,414, which describes a device for distributing a plurality of fluids comprising: a plurality of ports for receiving a plurality of fluids; a channel having two or more valves coupled to said plurality of ports; a first port coupled to said channel adapted to transfer fluid from said plurality of ports to a processing module, and to receive fluid from said processing module; a pump for controlling the transfer of fluids to the processing module; and a control module including an algorithm, the algorithm having variable inputs which control opening and closing of the valves and one or more of the temperature, pressure, volume and processing time of fluids transferred to the processing module.

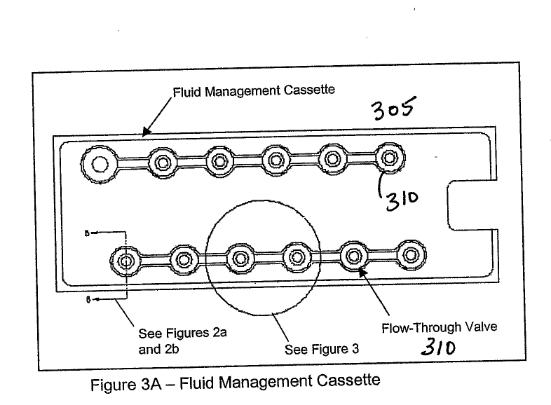

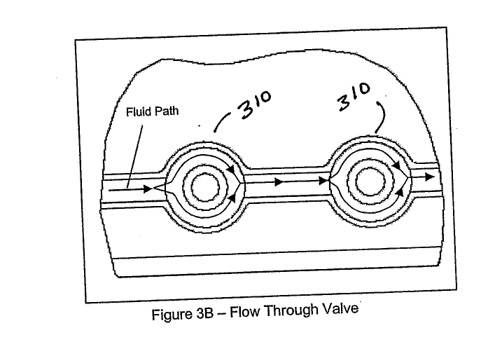

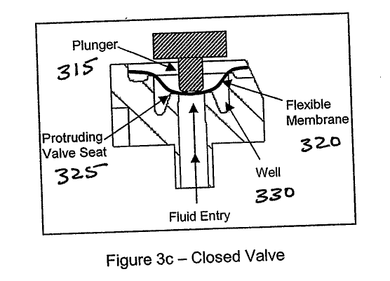

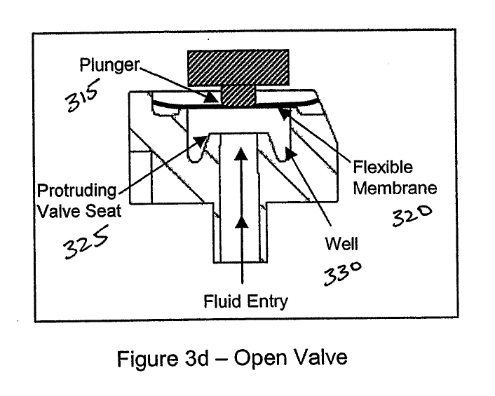

The embodiments illustrated in FIG. 3 are directed to a fluid management cassette, which includes a plurality of flow-through valves that control various fluid pathway communications. The fluid management cassette thus executes the instructions for regulating the supply of buffers, enzymes, sterile air and water, from the systems fluid reservoirs. As shown in FIG. 3A, the fluid management cassette 305 comprises a housing, preferably of plastic or another lightweight chemically resistant rigid material, including a plurality of fluid and air conduits, that terminate in one or more valves 310. The circumscribed area is presented in expanded view in FIG. 3B. In FIG. 3B the fluid path through the valves is illustrated. In FIG. 3C a valve is shown in cross section, in the closed position. Valves include a valve body having a valve seat 325, and a substantially flexible membrane 320 that is engaged and deformed by a plunger 315. Valve operation is performed by positioning the flexible membrane 320 over the valve seat 325 using a linear plunger 315 to open or close the valve and permit fluid pathway communication. The flow-through valve features a well that diverts fluid flow around the protruding valve seat 325 without flow interruption. This flow-through well design prohibits the trapping of fluids in dead areas and permits improved flushing of the fluid pathways. In FIG. 3D the valve is shown in cross section in the open position. The flexible membrane is no longer articulated against the valve seat, and fluid flows into the well.

### Processing and Expressor Bags

Flexible processing chambers (bags) for processing biological cells in a fixed volume centrifuge, and methods for use of such processing bags, e.g., by centrifugation, are known in various forms. For example, PCT patent application PCT/US98/10406 describes a flexible cell processing chamber having a rotating seal to keep the contents of the chamber sterile during processing (see also U.S. Pat. No. 5,665,048. Flexible processing chambers advantageously are disposable and thus suitable for single-use sterile applications.

For certain applications, such as blood processing including blood component separation, enzymatic conversion of blood type, and pathogen inactivation of blood components, it is desirable to remove portions of material separated by process, both light material and/or heavy material. Simultaneous processing of multiple separated material from the processing bag reduces the time and expense required to perform such applications. U.S. Pat. No. 7,074,172, describes an exemplary processing bag and its methods for use. It describes a flexible centrifugal processing bag for use with a component separator system for separating the components of a mixed material, said processing bag comprising: a hub having a central axis; a first port for receiving said mixture, wherein said first port includes an outlet positioned within said processing bag and spaced apart from the central axis a first distance; a second port having a second port inlet positioned within said processing bag and spaced apart from said central axis a second distance, wherein the second distance is different from the first distance, said second port for directing a separated material collected from said second port inlet out of said processing bag; and a third port having a third port inlet and a first filter having an inlet portion and an outlet portion, wherein said second port inlet is positioned adjacent said inlet portion of said first filter and said third port inlet is positioned adjacent said outlet portion of said first filter. In currently preferred embodiments, the processing bag includes a plurality of accordion-like partitions, that allow greater expansion of the bag when processing larger volumes.

FIG. 4 is a schematic drawing directed to a centrifuge processing bag. In a currently preferred embodiment, the processing bag is made of PVC and contains one or more raised surfaces in the bag, preferably composed of the same PVC material (for example) as the bag, and acting as a sealing surface and a mechanical strain relief.

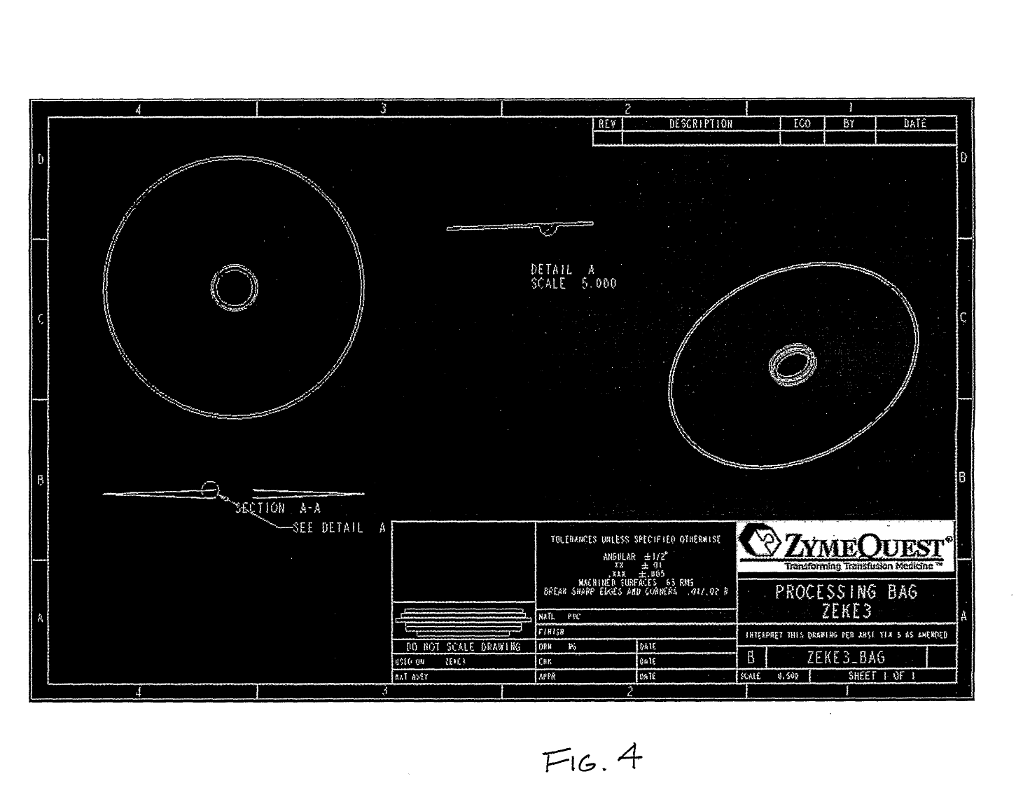

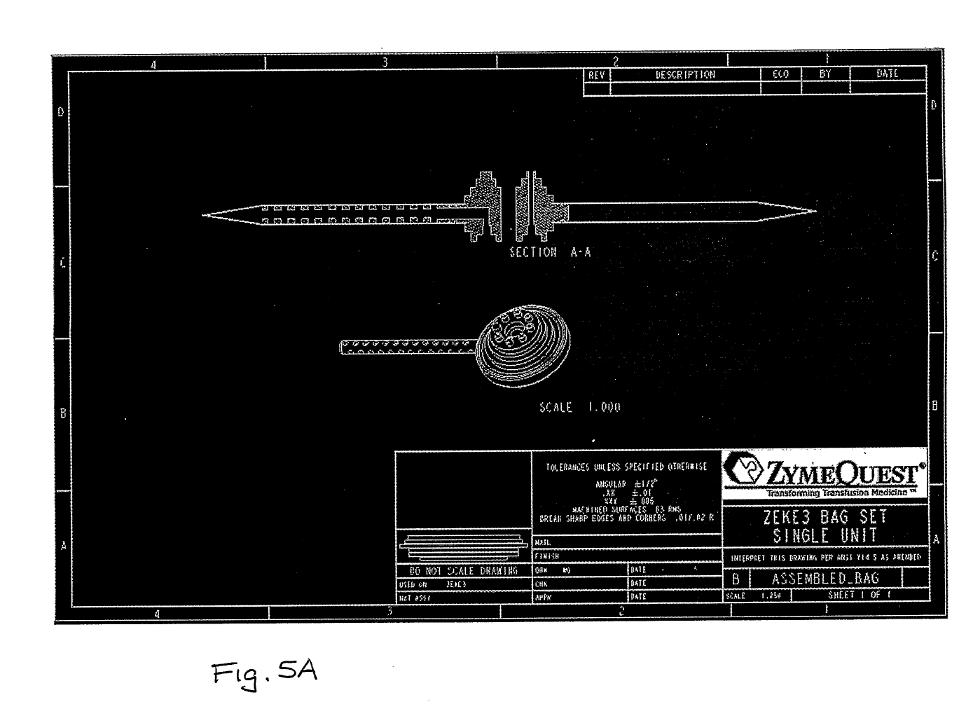

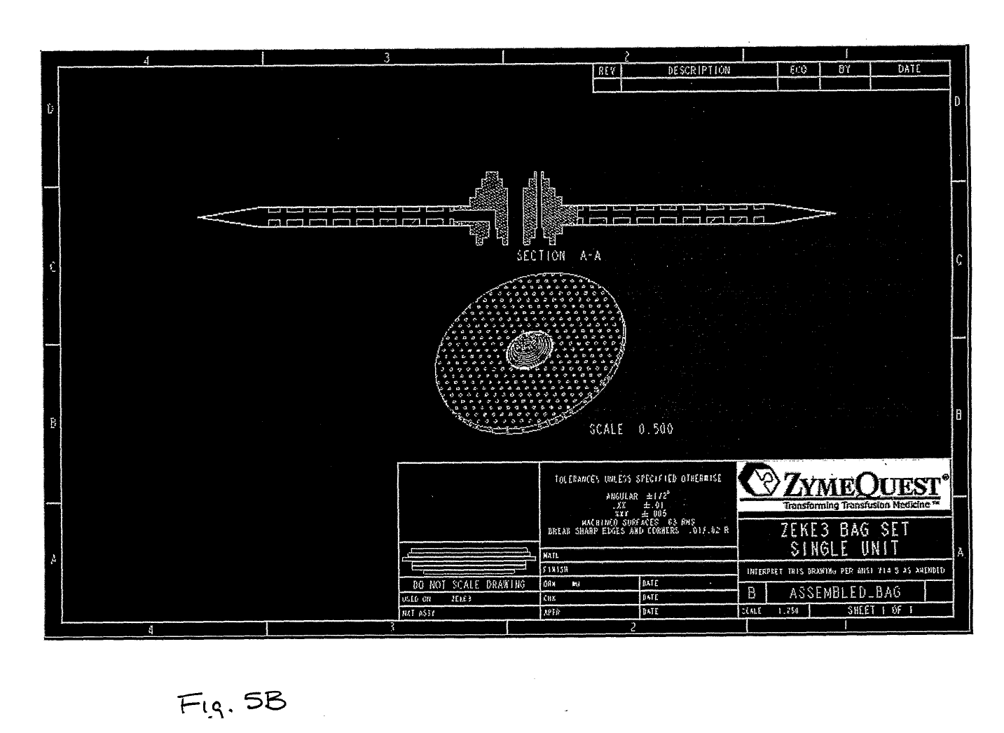

FIG. 5A is a schematic drawing directed to a modification of the processing bag design. Shown is a tube apparatus, which utilizes tubing preferably made of a rigid/high-durometer plastic material that can withstand the forces of blood product centrifugation, which attaches to the processing hub (or the centrifuge rotor in certain embodiments). The tube has a plurality of apertures along its sides, and the tube apparatus is itself inserted into and surrounded by the processing bag, protruding radially from the hub inside the bag lumen to about the internal periphery of the processing and/or expressor bag, and functions to maintain an open pathway and allow improved fluid flow into the bag.

FIG. 5B is a schematic drawing directed to a modification of the processing bag design. Shown is a bag apparatus having a plurality of geometric features adapted to the luminal walls of the bag. Preferably the geometric features and utilize an open cell foam. The geometric features provide the bag with improved resiliency, and prevent the sides of the bag from collapsing, thereby allowing fluid to pass through the bag with less resistance or flow restriction, between the bag lumen and the port on the hub to which the bag is secured.

FIG. 5C is a schematic drawing directed to a modification of the processing bag design. Shown is a bag having a plurality of integral raised features, for reinforcing the walls of the bag and preventing the walls of a processing and/or expressor bag from collapsing when centrifugal force is applied to the bag, and thus, improving fluid flow during device operation. In particular, this embodiment includes integral geometric raised features in the internal luminal surface of the bag. The integral raised geometric features are preferably made from the same or similar material as the bag. The apparatus allows the maintaining of an open fluid pathway within the bag, in the presence of high pressure air on the exterior of the centrifuge processing bag. The integral raised geometric features may be provided in any local area of the bag, from the periphery to the center hub, and preferably are concentrated in areas that are subjected to increased forces when the bag is rotated.

### Multi-Lumen Rope

As indicated previously, the basic structure of the centrifuge apparatus includes a bag set. This may also be referred to as a self-contained fluid retentive centrifuge cassette or rotor which is mounted on an inner-rotatable chuck. The bag set has fluid input and output coaxially and fixedly attached to the axis of the cassette. As shown, in our U.S. Pat. No. 7,008,366, the cassette is mounted on the chuck such that its rotation axis is coaxial along common axis. Thus, as the chuck rotates, the fixedly attached tubing co-rotates therewith. There is a length of the tubing which extends axially outwardly from the area of the fixed attachment. The length of tubing is curved axially backwardly toward and extends through a radially outer, separately rotatable pulley which rotates, by virtue of a gear train interconnecting the pulley and the chuck at a speed of 1X RPM while the chuck rotates at a speed of 2X RPM. Reference is made to U.S. Pat. No. 5,665,048 regarding the operation of the chuck and pulley arrangement. In operation, as the pulley rotates, the backwardly curved length of the tubing is rotated around axis at a rate of 1X RPM while the fixedly attached end of the tubing is actually rotated at a rate of 2X RPM. This phenomenon is generally known in the art as enabling the tubing to avoid twisting around its axis even as the cassette and the chuck forced the tubing to be axially rotated. A fuller description of this phenomenon is described in U.S. Pat. No. 5,665,048 as well as in U.S. Pat. No. RE29,738 (U.S. Pat. No. 3,586,413).

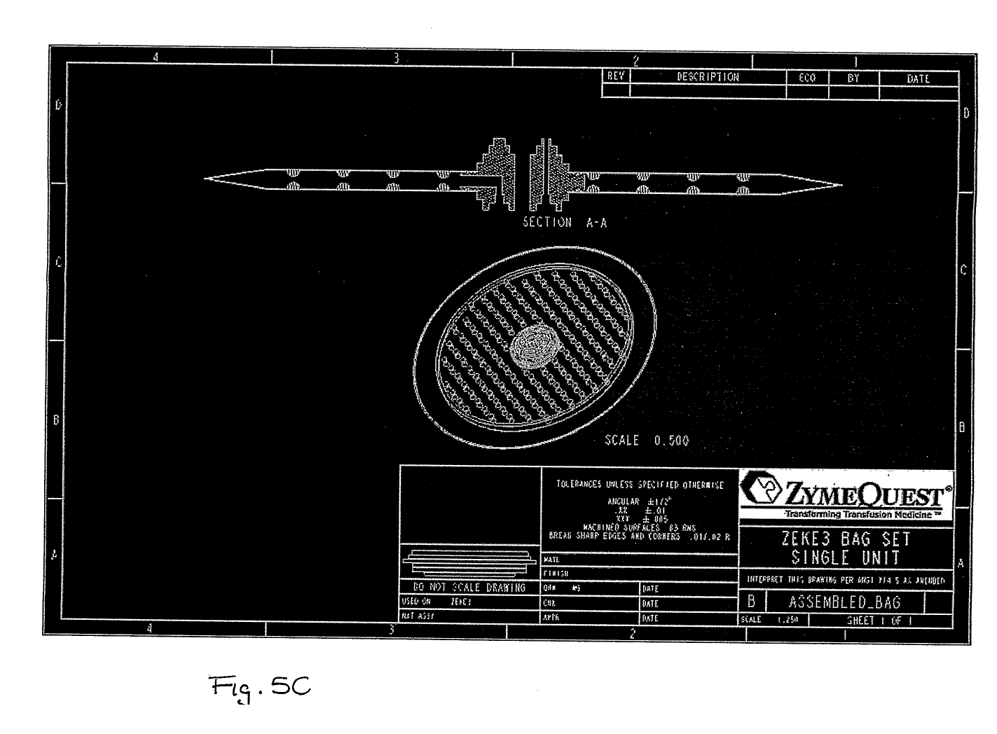

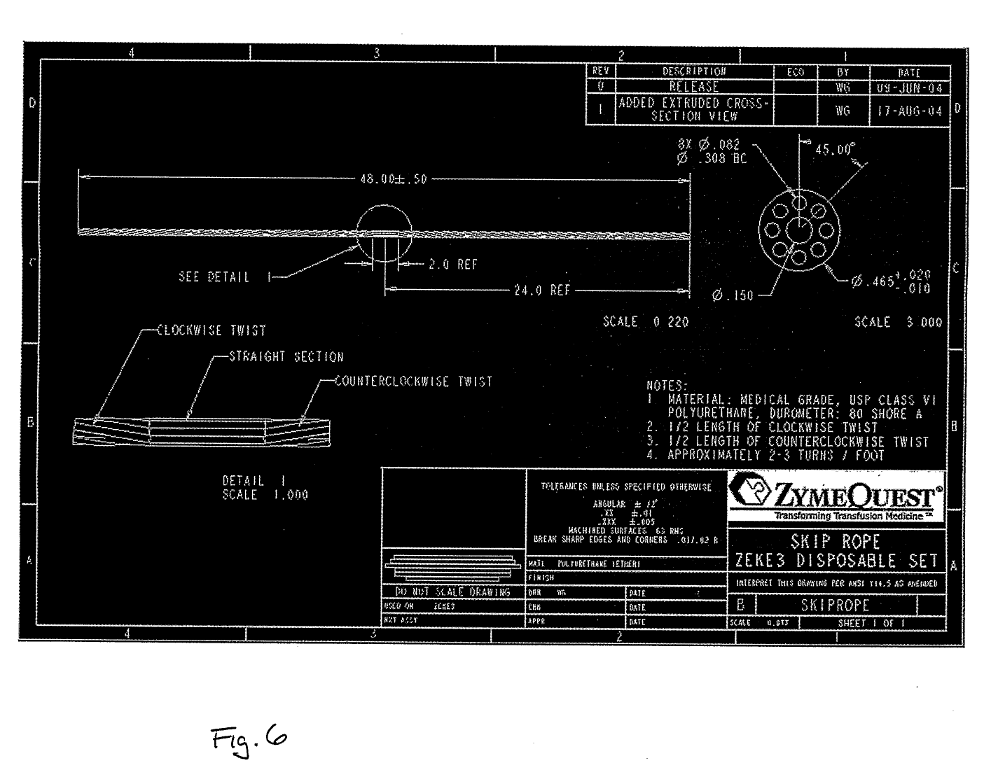

FIG. 6 is a schematic drawing directed to a multi-lumen tube corresponding to the “skip-rope” 120 (discussed above and illustrated in FIGS. 2A-2F). The multi-lumen 122 tubing (umbilicus) is used to carry fluids from the output of the system's supply module, i.e., from a static source manifold into one or more processing bags that are contained in the centrifuge. In particular, according to some embodiments, the umbilicus follows a specific trajectory from the static source manifold to the port in the centrifuge, and is supported by a rigid arm (see FIGS. 2A-2F). The umbilicus preferably may include a twist along a portion or all its length (e.g., to increase its structural rigidity). The support arm is geared to the centrifuge drive system as described above, which allows the umbilicus to spin in the same direction as the centrifuge at like or unlike speeds.

The multi-lumen rope may preferably be a twisted assembly of individual tubes, and more preferably is a single extruded member comprising a plurality of individual integral channels (i.e., a multi-lumen rope). An exemplary multi-lumen rope is manufactured from medical grade polyurethane, and has about 2-3 clockwise or counterclockwise turns per linear foot of length.

### Software/Electrical

The system described includes various electrical systems, processing devices and hardware, and instruction sets, e.g. software and firmware, for controlling the system. Exemplary and nonlimiting systems are described in Examples 1-3.

### EXAMPLES

### Example One

### Electrical Specification

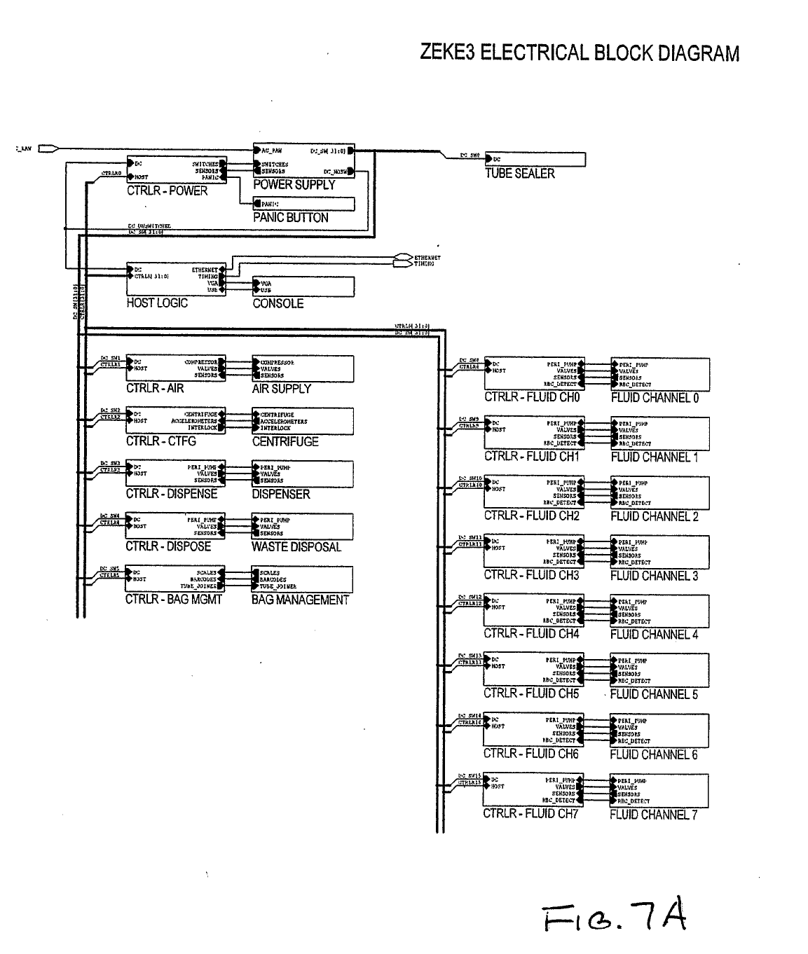

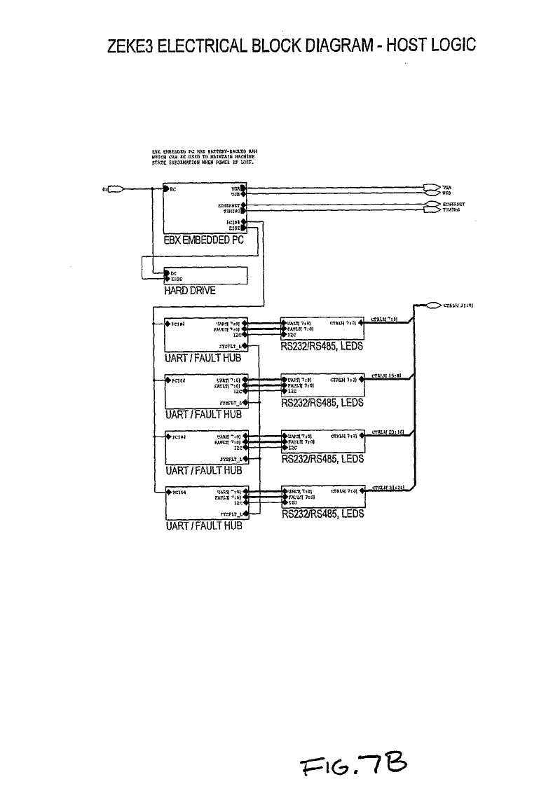

The following describes an embodiment of an exemplary electrical specification, which may be used with a centrifuge system according to one or more of the embodiments (discussed above), for processing one or more quantities of blood. FIGS. 7A and 7B represent an electrical block diagram, and host logic, respectively, for such a system. The following specifies the electrical and operator interfaces, power consumption, and electrical subsystems for embodiments of blood processing system. Each subsystem has its own detailed electrical specification.

### Electrical Block Diagram

The electrical block diagram is shown in FIG. 7A. The electrical system consists of a power supply, a host logic assembly, an operator console, and a number of subsystem controllers which interface the host logic assembly to the machine hardware.

The power supply takes in AC power and distributes DC power to the rest of the machine. An emergency disconnect, or “panic” button on the machine allows the operator to directly cut power to dangerous subsystems such as the centrifuge, the peristaltic pumps, or the air compressor.

The host logic assembly houses an embedded PC with up to four identical PC104 expansion boards (Zymequest, Inc., Beverly, Mass.). The expansion boards provide UART serial communications between the host PC and the subsystem controllers. The expansion boards are interconnected to provide a system Fault Hub which links the host and all controllers together so that they can mutually signal or detect a system fault. This is discussed more fully in Example Three below.

The subsystem controllers provide the host PC with a distributed software control interface to the machine hardware. The distributed control approach provides several benefits. A primary benefit is simplified cabling within the machine. Each controller is located as close as possible to the machine hardware which it controls. This keeps the large number peripheral component wires short and easily managed. Each controller has only two long cables routed to it, a power cable and a communications cable, and these are standardized to reduce the overall number of system cable types. The long haul cables can be considerably long without affecting performance, which means that the machine can be virtually any size and the controllers can be located virtually anywhere.

Locating the controllers near the machine hardware improves EMC (electromagnetic compatibility). High-current and high-frequency signal wires are kept short so they radiate minimal EMI (electromagnetic interference). Very-low-voltage and high-impedance transducer signals are kept short so they pick up minimal noise. The long haul power and host communication cables are twisted-pair to reduce radiated EMI.

Another benefit is intelligent autonomous control. In the case where the host PC locks up or otherwise malfunctions, the controller can detect the host lockup or reject out-of range host control commands. If host communications are lost, the controller can take action to bring machine operations to a halt before resetting.

An additional benefit is the standard power and control interface. This simplifies the test stations in the manufacturing environment. Any standard PC can be used to provide the RS232 communications needed to test the controller, and any bench top dual DC power supply can supply the power.

The Fault Hub is the basis for the machine's electrical fault detection and recovery. Each subsystem controller is connected to a port of the Fault Hub.

The controller signals its state to the Fault Hub: Idle, OK, or Fault. The Fault Hub signals its state to the controller: Idle, OK, or Fault. Whenever any port of the Fault Hub receives a Fault signal, the Fault Hub signals Fault on all of its ports. This forces all controllers to perform a hardware reset.

The OK signal is repetitive and has time constraints. If the communications cable between the Fault Hub and the controller is unplugged or comes loose or provides intermittent connection, the time constraints of the OK signal will be violated and this will signal a Fault to the Fault Hub and all attached controllers. The time constraints of the OK signal will also be violated if either the Host or any controller resets spuriously.

Electrical Interfaces

This section describes the electrical interfaces of the cell processing device. The AC power input to the cell processing device is a universal power jack. This will accept worldwide AC power mains from 100 to 240 volts RMS at 47-63 Hz. A regional power cable with the appropriate plug for the locale is provided to connect the wall outlet power to the universal power jack.

AC Power Switch

This switch is in series between the AC Power Input jack and the Power Distribution Subsystem. Switching to the OFF position will cut off AC power from the Power Distribution Subsystem. Switching to the ON Position will connect AC power to the Power Distribution Subsystem.

AC Power Light

The AC Power Light, which is located in an easily viewed location, indicates whether the system is energized. The AC Power Light turns on when the AC Power Input is connected to a live AC power source and the AC Power Switch is in the ON position. The AC Power Light turns off if the AC Power Input is disconnected or AC main power is dead, or if the AC Power Switch is in the OFF position.

Ethernet Communications

The network communications port of the cell processing device is a standard RJ45 modular jack which provides an autosensing 10/100 fast Ethernet interface. The communications protocols supported by this interface are specified in the 000-0000 Zeke3 Software Specification, described in Example Two.

Timing Port

This interface can be connected to a logic analyzer during system development to verify that the host software is running correctly. It is not accessible to the operator during normal use of the machine during blood processing runs.

### Example Two

### Instruction Sets

The following describes an overview of one example of a software system,

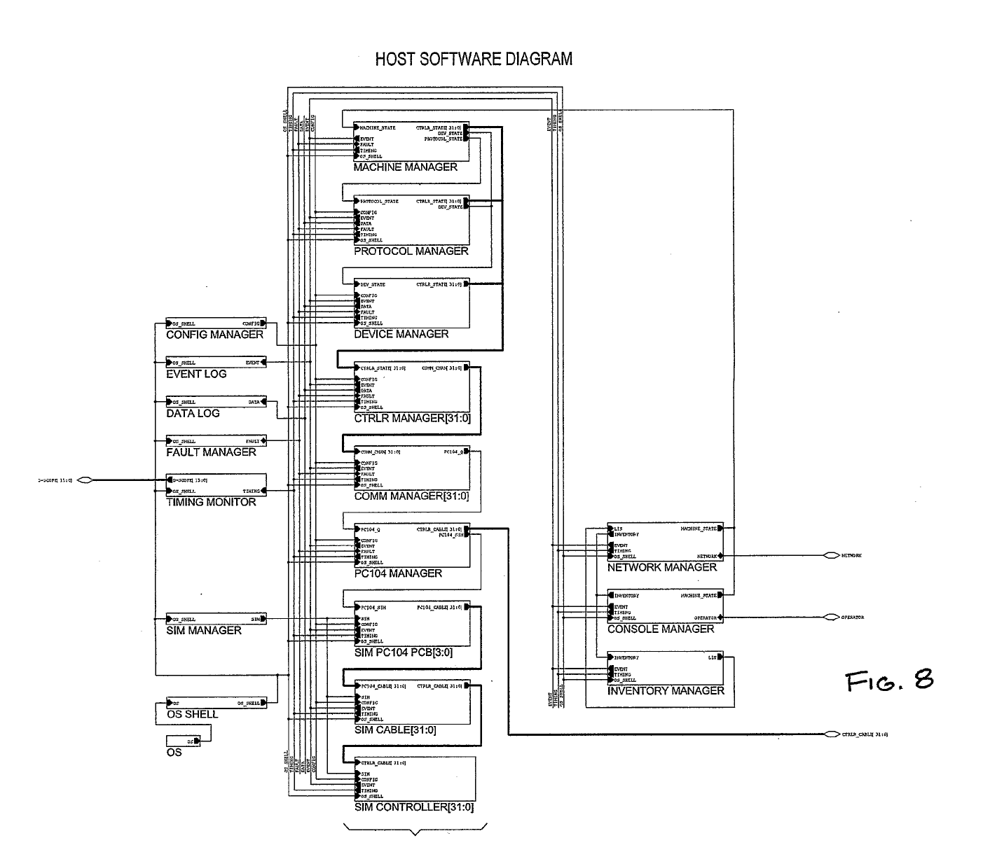

which may be used to control and/or operate the cell processing device according to one or more of the embodiments discussed above. FIG. 8 is a diagram of the host software architecture.

1 Overview

This document specifies the Host Software operating on a host hardware platform, for embodiments of the blood processing system according to the present invention. Other referenced specification documents provide focused detail of the software components.

1.1 Preface

This specification is written to provide the following:
Address safety concerns by applying fault detection at EVERY level of the software.
Section the software pieces so they can be implemented concurrently by contractors.
Keep the software as simple as possible, especially the interactions of the pieces.
Implement the software so it can run without machine hardware or the machine RTOS.
Guarantee system timing.

1.2 System Description

The Host Software provides a graphical user interface (GUI) for an LCD touchscreen. This provides instructions for loading the disposable set and displays progress during the blood processing. It also provides a diagnostic control interface for development or maintenance use. Text display is available in several different languages.

The Host Software may use Ethernet and TCP/IP networking protocols to interface to a central monitoring station. On top of these protocols, an LIS database is used to interface to a blood bank inventory database.

The Host Software uses a hard disk drive to store records of all completed or aborted blood processing runs. The hard drive is also used to store blood processing protocol definitions along with configuration and calibration information.

The Host Software uses an isochronous packet-based serial communications protocol to command and query the status of the embedded controllers which interface directly to the machine hardware such as sensors, motors, valves and the like.

The Host Software is implemented on top of a commercial real-time operating system (RTOS) to provide high reliability and guarantee that the machine operates in a safe manner.

The Host Software is segmented into components. Each component has one or more application programming interfaces (APIs). The APIs are designed to allow concurrent development of the components, so that more than one development team can be working to implement the components, and component implementation details can be changed as needed as long as the API remains unchanged.

Since machine hardware will not always be available to system developers, especially during hardware development, a simulation component is provided. This emulates the hardware operations with configurable degrees of fidelity.

Moreover, since the RTOS environment will not always be available to the system developers, all components may be implemented in a manner which allows them to be run on more than one type of operating system. This especially important during GUI development, so that the prototype software can be run on an ordinary Windows computer for customer feedback.

1.3 System Architecture

The Host Software Diagram is shown in FIG. 1. As shown, component and API names end in [n:0] to indicate that there are multiple copies of the component running, each dedicated to managing an individual resource.

A quick view of the Host Software diagram reveals three columns of components. The left column provides basic services which are easily ported to more than one operating system. These provide the foundation upon which the remaining system is built. The center column provides the machine control necessary to perform autonomous blood processing. The right column provides the external interface to the machine.

It is important to note which components are interconnected and which aren't. Each component is preferably designed to have a minimum number of inter-component dependencies. This simplifies the component and focuses implementation, making testing easier and quicker.

2 Basic Services

The basic service components isolate the operating system specific API from the higher layer components. Each basic service component provides a fixed API to higher layer components and may be implemented in more than one version to support more than one operating system.

2.1 Operating System

This is the lowest layer in the Host Software. It provides access to low-level hardware resources including the CPU, ROM, RAM, storage devices, display devices, network devices and the PC104 hardware expansion bus.

The release version of the Host Software uses (specify name here, i.e. VxWorks, QNX) as the RTOS. This provides deterministic scheduling of tasks using a pre-emptive priority algorithm. This ensures that the highest-priority task is always running. Tasks of equal priority are scheduled using a round-robin time-slice algorithm which ensures that each task has the same opportunity to run.

The development version of the Host Software replaces the RTOS with Microsoft Windows.

2.2 OS Shell

The OS Shell contains all of the Host Software references to the underlying operating system API and is re-implemented for each operating system. The OS Shell API provides higher layer components with a view of a generic operating system.

The generic operating system may provide one or more of the following services:
console inputs from a keyboard and a pointer device (such as a mouse or a touchscreen);
console text output;
high-resolution time stamp;
error logging;
primitive execution threads;
primitive execution critical sections;
primitive heap memory;
kernel services;
application management; and
fault detection and recovery.

2.2.1 Primitives

The console input/output services are provided for use during development. They are not the basis for the graphical user interface which is provided by the Console Manager component.

The high-resolution time stamp service returns a double-precision floating point number which represents the number of seconds since system startup. An underlying operating system call which counts very fine time increments on the microsecond or nanosecond order of magnitude provides the basis for the time stamp.

The error logging service allows higher layer components to indicate problems by logging an error code. The error codes are time-stamped and placed in a log file on the hard drive. In addition, a copy of the error code is provided as console text output.

The generic operating system provides flexible and extensible code execution in the form of a thread primitive. This thread primitive takes three main parameters when it is created; a task priority, a timer period and a callback function. The thread primitive will execute the callback function once every timer period from an underlying operating system task running at the specified priority. The intended method of managing the logic and resources of a thread primitive is a finite state machine.

The thread primitive has the limitation that the callback function must return before a new timer period begins. This means that the callback function must adopt a polling method of communications and control.

The callback timing limitation is actually the main advantage of the thread primitive. The developer is forced to implement the system components in a non-blocking manner which guarantees deterministic execution timing. The thread primitive monitors the execution time of the callback and generates an error if the thread violates its timing period.

Since the thread primitive is simply a periodic callback function and is not an underlying operating system task, advanced operating system blocking mechanisms such as semaphores and mutexes are avoided. This simplifies the portability of the OS Shell component.

Multi-threaded applications inevitably need to share data structures between threads. A race condition occurs when two threads simultaneously update and access the same data structure. It is this problem which causes the need for a controlled method of execution blocking.

The sole blocking primitive provided by the generic operating system is a critical section. Some underlying operating systems will implement this primitive directly, others will use a semaphore or mutex to provide the blocking.

The critical section uses a lock function to allow a thread primitive to begin executing a section of critical code. The lock function prevents other thread primitives from simultaneously entering the section by temporarily blocking them inside the lock function. When execution of the critical code section is complete, an unlock function is used to unblock any blocked thread primitives. Critical sections of code are meant to be as short as possible so that blocking will be kept to a minimum.

Memory fragmentation is a common problem with heap memory which can lead an application to crash. Heap memory is allocated dynamically during runtime, used for some duration, and eventually freed for reuse. Fragmentation occurs when randomly located small pieces of freed memory can't be combined together to satisfy a request for a contiguous larger piece of memory. In this case, the request would fail and this could lead to an application crash.

It should be noted that the heap memory fragmentation problem can often be avoided completely by using static memory allocation instead of using heap memory. The static allocation method reserves memory at application compile time instead of at runtime. The potential drawback to this method is that this memory can't be returned to the operating system for reuse until the application quits.

The OS Shell component solves the heap memory fragmentation problem with a two-stage heap memory allocation scheme. The primitive heap layer directly uses the underlying OS to allocate and free memory blocks. The higher heap layer is discussed in the kernel services section.

2.2.2 Kernel

The OS Shell component provides a kernel layer which builds on the OS Shell primitives. The kernel services offer much greater flexibility and utility than the primitives. The kernel services are:
heap management;
linked list management;
circular buffer management;
task management;
message queue management; and
text window management.

The kernel heap layer uses the primitive heap layer during application initialization to allocate a series of relatively large memory pools. Each of the pools are divided into a number of fixed-size free blocks. Each memory pool is divided into a different free block size from the other memory pools. The memory pools remain allocated until application cleanup. This defers the fragmentation problem to free block management.

Application memory allocations are satisfied in a two step process. First, the pool with the smallest free block size which is at least as big as the allocation size is selected. Then, one of the fixed-size free blocks from the pool is allocated and provided to the application. When the fixed-size block is later freed by the application, it is returned to the same pool it was allocated from, as a free block.

Memory fragmentation never occurs because the fixed-size free blocks of the memory pools are never subdivided and do not need to be recombined. The order in which the memory pool free blocks are allocated and freed does not lead to fragmentation.

The linked list kernel service provides higher layer components a simple reliable method for managing lists of objects. The service allows objects to be inserted or removed from a list. An object can be placed into a specific location in a list by providing a sorting algorithm. A list can be traversed by moving from an object to the list's next or previous object. A list can be split into two lists and two lists can be merged into one.

The circular buffer kernel service provides a simple reliable method for temporarily holding data which is created at a time different from when it is needed. A good example would be an interrupt service routine which writes incoming serial communications data into a buffer so it can be read later by an application. The circular buffer kernel service allows buffers of any size (up to a limit), and allows simultaneous read and write operations on the buffer. Data which has been read out of the buffer can be re-read as long as it is not overwritten by the circular reuse of the buffer.

The kernel task management service is much more flexible and full-featured than the thread primitive. It provides event processing tasks, event timers, and task schedulers.

Any number of task schedulers can be created. Each one creates its own dedicated thread primitive. When a task is created, it is assigned to a task scheduler.

A kernel task is much like a thread primitive as it is created with a priority, a timer period, and a callback function. The difference is that the callback function is provided an event which it must process.

Kernel task events have two possible sources. One source is kernel tasks. Kernel tasks may send events to other tasks, or to themselves. The other source is a kernel task timer. When a kernel task timer expires it sends an event to a task. Kernel task timers may be configured for single-shot or repeating expiration.

A kernel task scheduler manages kernel task timers and kernel tasks. The scheduler itself is a thread primitive, which means it runs as a callback on a periodic schedule. When the scheduler runs, it examines all its task timers and sends events for any timers, that have expired. Sending an event will cause a task to be moved from the scheduler's idle list to its ready list. The ready list is sorted by priority. After managing the timers, the tasks in the ready list are processed by calling each task's callback to process the task's pending events. The scheduler's job is done when there are no more tasks in its ready list.

The message queue kernel service provides more flexible inter-task communications than simple task events. It allows any number of sending tasks to send any number of messages of any format to a receiving task for processing. Sending a message causes an event to be sent to the receiving task, to indicate that the queue is not empty.

The text window kernel service utilizes the OS Shell console input/output services to provide a much more flexible and full-featured console interface. It derives all of its functionality from a configurable base window object and a kernel task which automates console text output updates and assigns console input to base windows.

The base window consists of two rectangular regions, the border and the center. The border can be blank, single-lined, or double-lined. The center is the region enclosed by the border. The foreground and background color of each region can be set independently. The base window can be located anywhere in the console, and the base window size can be any size which fits in the console. The base window also has a text string. The text string is displayed in the center if the border is blank, or in the border otherwise. Another attribute of a base window is whether it is visible or not.

A base window can also have a stack of other base windows on it. This provides the hierarchical order in which the console output is updated. First the root window is updated, then each of its stacked windows is updated, and each one of those stacked windows can have any number of stacks which are updated using recursive logic. If a base window is set to be not visible, all windows in its stack are also not visible.

The base window has four callback functions which are used to further customize the base window. One callback signals the opportunity to change the base windows parameters such as color, position, size, or visibility. The other three are used to send keyboard or pointer information to the base window which has focus.

Base window focus is selected when a pointer event such as a mouse button or touchscreen press occurs. The console location of the event is compared against the window stack hierarchy. The root window gets the focus first. Then each of the windows in its stack is checked to see if the pointer location is in the window region. If so, that window gets the focus. This search repeats recursively as with the update logic.

The text window kernel service provides other more specialized types of text windows. These include a popup window, a button window, a datum display window, and a progress bar window. A frame window type is provided which manages lists and hierarchies of button windows, each of which gets a large portion of the console output area for customized display when a button is selected.

2.2.3 Application Management

The OS Shell manages the three phases of system operation; initialization, runtime, and cleanup. These distinct phases always occur in the stated order.

The OS Shell initializes each of its subcomponents in an organized manner, checking all along to prevent recursion. Initializing a subcomponent more than once will cause an error which will crash the system. After the OS Shell component is initialized, it calls a fixed-name function to initialize the application. This is much like the main( ) function which is used in ANSI C as the starting execution point for a program.

When the application initialization function returns without error, the OS Shell component enters its normal runtime phase. It will stay in this phase until the system detects an error or a subcomponent signals a shutdown.

Then the cleanup phase is entered. First a fixed-name function is called to cleanup the application. This function is used to safely halt any ongoing machine operation and release all acquired OS Shell component resources. Then the OS Shell subcomponents clean themselves up in the reverse order from which they were initialized.

The OS Shell cleanup functions log warnings any time a resource is found which should have been cleaned up already. These warnings indicate that one or more higher layer components executed an incomplete cleanup.

2.2.4 Fault Detection and Recovery

The OS Shell component is in a unique position to act as the final line of defense against uncontrolled system crash. This is because it control the three phases or system operation, and because it provides the thread primitive and kernel tasks which run the system.

Every function which implements the OS Shell component returns an error code value which is zero for normal operation and nonzero to indicate an error. This applies also to the callback functions of the OS Shell API. Meaning, higher layer components which use the OS Shell are forced to supply an error code with every function call.

Each error code is unique. This makes it easy for developers to very quickly diagnose what has gone wrong when an error code is logged, by finding the single place in the source code which can generate the error.

After each function call, the resulting error code is tested against zero. If it is nonzero, sometimes the unique error code indicates a condition which can be tolerated and recovered from. In this case, a remedy is executed and the system continues operation. If not, the error code is passed up through the chain of function calls until it is trapped and remedied at a higher layer or it is detected by the OS Shell.

When the OS Shell detects an error code, it moves swiftly to the cleanup phase which will attempt by the normal method to halt any ongoing machine operation. The error code could indicate that an important data structure has become corrupted, and this could interfere with normal cleanup. If this happens, the system may crash.

In this case, the hardware PC104 Fault Hub circuit will detect the lack of system action and will halt the machine automatically

2.3 Fault Manager

The Fault Manager component provides a service which other components use to share fault status information.

The Fault Manager is unlike the OS Shell fault detection and recovery, which reacts only in the case of an unrecoverable error. The Fault Manager is designed to provide system response to dangerous fault conditions such as centrifuge over speed or harsh vibration, unusual lagging centrifuge or peristaltic motor movement, or overpressure in the air system.

The Fault Manager acts like one would expect an Emergency Stop Button to act. When any component signals a fault, all components receive the fault signal and each component reacts by halting all ongoing machine operation.

The Fault Manager operation is modeled after the hardware PC104 Fault Hub operation. The Fault Hub is an advanced version of a basic watchdog circuit.

A basic watchdog circuit controls the reset input to a hardware logic circuit. The logic circuit must periodically restart the watchdog circuit's timer to prevent the watchdog timer from expiring. If the watchdog timer were to expire, the watchdog circuit would signal a reset to the logic circuit.

The Fault Hub extends the watchdog concept by linking numerous watchdog circuits together so that when any of the watchdog timers expires, reset is signaled to all of the logic circuits.

A component begins its interaction with the Fault Manager by registering itself and providing a deadline period. After registration, the component must report its fault status periodically. This restarts a deadline timer which the Fault Manager maintains for the component. If a component's fault status report is not received before its deadline timer expires, the Fault Manager enters the Fault state.

Each time a component reports its fault status, it receives a report of the Fault Manager status. When the component receives a report of the Fault state, it must react by halting any ongoing machine operation and then report back to the Fault Manager that the fault was successfully handled.

An important part of the Fault Manager operation is that it runs in its own dedicated OS Shell thread primitive at the highest priority of all machine control tasks, and it does not make use of the kernel task services. This guarantees that no other thread primitive or kernel task which is deadlocked or stuck in an infinite loop can prevent the Fault Manager from performing its deadline timer maintenance.

When the Fault Manager enters the Fault state, it starts a shutdown timer. If then begins waiting for each registered component to report that it has handled the fault. If all registered components handle the fault before the shutdown timer expires, the Fault Manager stops the shutdown timer and exits the Fault state.

If the shutdown timer expires, the Fault Manager reacts by retiming an error code from the thread primitive, which will cause the OS Shell to move into its cleanup phase.

2.4 Timing Monitor

The Timing Monitor component provides a service which other components use to output a coordinated set of signals to a hardware output port which can be connected to an oscilloscope.

The core of the safety management system for the Host Software is based on detection of timing violations.

The OS Shell thread primitive verifies that its callback function completes its execution within its allotted period. If it executes for too long, the thread primitive returns an error code which will cause the OS Shell to transition to its cleanup phase, which will in turn force all components to halt all ongoing machine operations.

The Fault Manager provides a service which allows the system to react to detected unsafe conditions. If the system response does not complete within a timing deadline, an error code is generated which will cause the OS Shell to transition to its cleanup phase.

It will be seen in section 3.2.2 that reliable timing of the serial communications protocol is critical to its operation. This is because the slave controllers at the far end of the serial connections use the communications stream as an input to the PC104 Fault Hub. If a communications timing violation occurs, a slave controller will signal a fault to the Fault Hub, and this will halt all ongoing machine operations.

The Timing Monitor is used during Host Software system development to view the timing of the system. It can be used to determine how much margin there is in the timing. Where margins are too thin, the Timing Monitor can be employed to determine which portions of the software need performance optimization.

The Timing Monitor provides a number of functions which allow direct updates to bits on a CPU output port. The oscilloscope interfaces directly to this output port. Timing Monitor functions calls get placed in key locations of the system source code to generate the coordinated set of signals on the output port.

2.5 Event Log

The Event Log component provides higher-layer components a uniform method for logging information about system events. The events are logged to a file on the hard drive.

Any number of event log files can be generated simultaneously. This allows components to maintain their own event logs without cluttering up a system event log with verbose amounts of information.

The Event Log component provides a runtime configurable event filter which can prevent insignificant events from being recorded in the log file. A component can be implemented at compile time to generate much more information than is normally needed, and at runtime only significant events could be logged. If a problem with the component needed to be investigated, all more or all of the events could be logged to provide better insight into the problem.

2.6 Data Log

The Data Log component provides an standard extensible method for logging system variable information. The data is logged to a file on the hard drive.

Any number of data log files can be generated simultaneously. This allows components to maintain their own data logs without cluttering up a system data log with verbose amounts of information.

The Data Log component interacts with other components to receive their data at any sample rate, and log it to die hard drive at a configurable constant recording sample rate. It is intended that the recording rate is significantly slower that the incoming data rate.

The Data Log manages each data file in two phases. The first phase is a registration phase. In this phase, other components register the structure of the data that they want to log. Each structure is comprised of a set of primitive data types like floating point or integer. Registration continues until a component commands the Data Log to commence data recording.

At the end of registration and before data recording, the Data Log service writes the registered format information into the Data Log file so that the data recording format can be interpreted. This method allows the Data Log file to contain any combination or permutation of the primitive data types.

During the recording phase, each component's data stream is handled one primitive data type at a time. Each incoming datum is tested to determine the minimum, maximum, and average value of the datum during each Data Log recording period. When the Data Log recording sample is written to disk, these three statistics are written for each datum. This triples the disk file size but effectively represents the datum range during each recording period.

2.7 Configuration Manager

The Configuration Manager component provides a standard method which other components may use to read in configuration parameters from a file on disk. Any number of configuration files can be used.

The Configuration Manager reads in a formatted text file which has comments or configuration values on each line. Each configuration value consists of a name, a data type, and a data value. Components query the Configuration Manager by providing a value name and get in response the data type and data value.

2.8 Simulation Manager

The Simulation Manager provides a framework within which the machine hardware can be emulated with realistic system timing. It provides for the simulation components the management of virtual time which progresses in lock step with real time.

As an example, take the serial transfer of a byte of information. At a coarse degree of fidelity, this could be simulated by copying a byte from one C-variable to another. The real machine time that it would take to do this is very tiny, far faster than a serial byte transfer would take at 9600 baud, which is 1042 microseconds.

The Simulation Manager allows the developer to extend the basic byte copy model by adding a virtual time delay of 1042 microseconds after the byte copy.

3 Machine Management Services

The machine management services provide a layered hierarchy of software components which directly control or simulate the machine hardware, automate the hardware control according to a predefined blood processing protocol, and provide an interface which allows operator control of the machine.

The bottom layers of the machine management services provide direct or simulated control of the machine's embedded hardware controllers. These embedded controllers each have two dedicated connections to the machine's PC104 I/O hardware. One connection is the PC104 Fault Hub safety circuit. The other is a full-duplex serial UART communications link.

The middle layers of the machine management services provide the isochronous serial communication protocol. This protocol reliably transports formatted messages between the Host Software and each embedded controller. Each embedded controller is sent formatted command messages and in turn sends back formatted status messages to the Host Software. The command messages are translated by the embedded controllers into hardware control. The status messages indicate to the Host Software the result of the command.

The top layer of the machine management services operates in one of two control modes, automatic or manual.

In the automatic control mode, a predefined blood processing protocol is executed. The protocol consists of a list of machine instructions which are performed in sequential order. The operator interface allows the following protocol control options: start, pause, resume, or abort. Once a protocol has started the operator must either abort the protocol or wait until it finishes normally before the manual control mode can be initiated.

In the manual control mode, the operator interface allows control of individual hardware elements. For example: the fluid management pathway valves can be opened and closed, the centrifuge speed can be changed, or a barcode can be read. The manual control mode is intended for testing or diagnostics and is limited to special operators such as machine service or machine manufacturing personnel.

3.1 PC104 Manager

The PC104 Manager component is the lowest layer of the machine management services hierarchy. It provides the Host Software access to each embedded controller's serial link and gives a read-only view of the Fault Hub circuit status.

The PC104 Manager component is the only one which communicates directly with the PC104 interface. If the PC104 hardware is missing, its operation and the operation of the embedded controllers is simulated.

3.1.1 Serial Link I/O

Each controller's serial communications link is represented to the next higher of the layer machine management services as a pair of unformatted byte streams. This means that the bytes are processed without recognizing any byte patterns such a packet or a message.

The downstream link to the embedded controller is a is represented by a transmit queue. The upstream link from the embedded controller is represented by a receive queue. Only the Communication Manager is granted access to these queues.

Whenever any of the transmit queues is not empty, the PC104 Manager copies the byte stream data from the queue to the appropriate PC104 registers.

Whenever any of the PC104 hardware receive buffers is not empty, an interrupt is generated. The Host Software responds to the interrupt by copying the byte stream data from the hardware receive buffer (via PC104 registers) to the appropriate receive queue. This in turn alerts the higher layer that the new stream data needs to be processed.

3.1.2 Read-Only Fault Hub Status

The fault hub status is represented to the higher machine management services as a read-only 32-bit integer. Only the Fault Manager component is granted access to the fault hub status.

Each bit of the 32-bit integer represents the state of an individual controller's Fault Hub signaling. A 1 bit represents the MFLT state which indicates that the Fault Hub is forcing all embedded controllers to reset. A 0 bit represents the MOK state which is normal.

The status readout logic in the Fault Hub hardware is a synchronous circuit which stores the state of all Fault Hub ports when the first port enters the MFLT state. These state values are read via PC104 registers to form the read-only 32-bit status integer.

3.1.3 Simulation

Hardware simulation is provided by three lower-layer software components. These simulate each PC104 I/O board, each cable, and each embedded controller.

The PC104 I/O board; hardware is simulated at the PCB level to mimic the timing delays incurred in the UART buffers and to emulate the serial baud rate. Simulations of the PC 104 registers provide the interface to the higher layer. Simulations of the serial link I/O and Fault Hub signals provide the interface to the cable simulators.

The system cables are each simulated individually to mimic physical plugging and unplugging and allow study of bit errors due to electrical noise. Modified versions of the higher layer cable simulations are passed down to the embedded controller simulators.

Each embedded controller is simulated individually to mimic the message processing delays and provide appropriate status messages. The embedded controller simulation can include simulation of controlled hardware responses such as motor inertia or solenoid switching delay.

3.2 Communication Manager

The Communication Manager implements the Isochronous Serial Communications Protocol for each controller. The protocol specifies communication between the Host Software and one controller. The Communication Manager implements the protocol independently for each controller.

The interface to the next higher layer is a pair of message queues for each controller. One is a transmit queue which holds formatted command messages destined for the controller. The other is a receive queue which holds formatted status, messages returned by the controller, one for each of the original command messages. Only the Controller Manager is granted access to these queues.

The interface to the next lower layer is the PC104 Manager. For each controller there is a pair of unformatted byte streams; one downstream to the controller, one upstream from the controller.

The Host Software is the master of communications. It expects from the slave (controller) a prompt response to every command packet, in the form of status packet. It expects commands will not be parsed and acted upon out-of-order. It expects that the slave can transmit and receive packets at full speed simultaneously (full-duplex communications) and continuously. It expects that the slave will never send an unsolicited packet to the Host Software.

The Isochronous Serial Communications Protocol provides two services vital to machine safety:

1) reliable data transfer

2) fault detection and recovery

The protocol specification document also details a set of basic command messages which all controllers must support, and the status messages which are generated in response. These include an initialization command which starts the controller's hardware Fault Hub signaling. Commands unique to each controller are specified in an addendum to the basic protocol specification.

3.2.1 Isochronous Definition

Isochronous refers to processes where data must be delivered within certain time constraints. For example, multimedia streams require an isochronous transport mechanism to ensure that video data is delivered as fast as it is displayed and to ensure that the audio is synchronized with the video.

Isochronous can be contrasted with asynchronous, which refers to processes in which data streams can be broken by random intervals, and synchronous processes, in which data streams can be delivered only at specific intervals. Isochronous service is not as rigid as synchronous service, but not as lenient as asynchronous service.

3.2.2 Reliable Data Transfer

The basic unit of data transfer in the Isochronous Serial Communications Protocol is a formatted packet of information. The packet format consists generally of four sections; a preamble, a header, an optional message, and acyclic redundancy code (CRC).

The preamble is a fixed byte pattern for all packets, and is used to signal the start of a new packet. The header specifies the packet type, the message length, and a sequence number. The optional message can be any byte pattern of the specified length (which is zero when there is no message). The CRC is a byte value calculated based on the byte values in the header and the message. The CRC is used to determine whether a received packet has been corrupted by noise during its transmission.

For each locally transmitted original MSG type packet, the protocol requires the remote end to send back an ECHO type version of the packet. An original MSG packet's message can be a Host Software command message or a controller status message. The ECHO packet has the same sequence number, message length, and optional message but will contain a different packet type field (ECHO) and CRC.

Upon receipt of the ECHO packet, the local end compares the original MSG packet's message length and optional message against the echoed copy. If they match, the local end sends a special AACK type packet to acknowledge that the correct echo was received. This indicates to the remote end that it may further parse and act upon the original MSG packet's message. If not, a NACK type packet is sent to indicate that the remote end must ignore its original MSG packet. An AACK or NACK packet has the same sequence number as the original MSG packet.

There is normally a six-packet transaction which is required to complete a communication between the Host Software and an embedded controller.

1: Host Software sends command MSG packet, controller holds this MSG until AACK or NACK is received

2: controller sends command ECHO packet

3: Host Software sends AACK packet, controller parses and acts upon the held command MSG

4: controller sends status MSG packet, which contains results of command MSG parsing and action

5: Host Software sends status ECHO packet

6: controller sends AACK packet

Abnormal transactions involving NACK packets or CRC failure are detailed in the protocol specification document. Error recovery entails resending the original command or status MSG packet. If the error condition persists beyond an isochronous deadline timeout, a fault condition is signaled.

The sequence field in the packet header is used only by the Host Software. The Host Software generates a new value in the sequence new each time it generates a new command. This is so that identical commands and their resulting status messages can be distinguished from each other. The sequence field is managed by the Controller Manager.

3.2.3 Fault Detection and Recovery

The isochronous nature of the serial protocol is used by both the Host Software and the controller to detect a communications fault. A communications fault is caused by either a software lockup or reset on the remote end, or by a hardware connection problem. Connection problems include data corruption due to electrical noise, intermittent connection, accidental or intentional disconnection, and failed or faulty communication component.

The Host Software expects that the six-packet exchange will complete within a time limit. If it doesn't, the Host Software assumes that the slave can no longer be controlled. The Host Software responds by reporting the fault to the Fault Manager, which will halt all ongoing machine operations.

The controller expects that the Host Software will always be sending commands to maintain communications. If the controller does not parse a new command within a time limit, the controller will signal a fault to the hardware Fault Hub, which will halt all ongoing machine operations.

3.3 Controller Manager

The Controller Manager provides two services to the Host Software:

1) automated periodic communication with the embedded controllers

2) a function call API which provides all of the direct machine hardware control and status queries

3) redundant safety enforcement for each controller

The Controller Manager interfaces with four of the Host Software components. The lower layer interface uses the Communication Manager to directly command and query the embedded controllers. The higher layer interface is shared by the Device Manager, the Protocol Manager, and the Machine Manager.

To prevent the three higher layer components from providing conflicting commands to the hardware, access to some of the API functions is granted to only one of the components at a time. To access these functions, a higher layer component must succeed in registering itself with the Controller Manager as the owner. If the registration fails, another component is already the owner. A component can relinquish ownership at any time.

3.3.1 Automated Communication

The Isochronous Serial Communications Protocol requires each embedded controller to parse and act upon new command messages at a periodic rate faster than that imposed by its timing deadline. Failure of the Host Software to send a new command before each deadline expires will be met with a hardware Fault Hub reset of all of the controllers.

The Controller Manager meets the periodic communication timing requirement by generating a continuous stream of controller status query commands. The result of these commands provides the Communication Manager with the data needed to maintain a comprehensive cache of the system hardware state. This status cache is updated in realtime.

3.3.2 Function Call API

The Controller Manager greatly simplifies the higher layer's communication with the embedded controllers by providing a function call interface to the Communications Manager.

For each type of controller, the Controller Manager provides a customized set of API functions which implement the unique commands available from the Communications Manager for that controller. API functions are also provides for the common controller commands.

For example, the Controller Manager would provide for the centrifuge controller a group of functions to set and read back the centrifuge speed, lock and unlock the access door, read the vibration level, and more.

As another example, the Controller Manager would provide for a fluid management controller a group of functions to change and read back valve states, read the hematocrit value in a given optic detection channel, deliver a specified fluid volume from a peristaltic pump, etcetera.

Without the Controller Manager, the higher layer's communication with the controllers would be packet-based. Each time the higher layer needed to change or query the controller's hardware state, it would need to build a packet of relevant information, send it to the Communication Manager, and wait until the Communication Manager received and forwarded the controller's status packet.

The packet exchange method is cumbersome, and it includes waiting for a relatively long time which is bounded but not deterministic. It also requires the higher layer to be intimately involved with the serial protocol's timing requirements.

Instead the Controller Manager allows the higher layer to make simple program function calls. These delay the higher layer for an insignificant amount of time. The automated communications feature of the Communication Manager completely isolates the higher layer from any timing requirements.

In general there are two types of API functions, command and query. Command functions are the ones which require ownership to access. Query functions may be accessed without ownership.

Query type functions return the relevant information from the Communication Manager's realtime cache of the hardware status. This bypasses the need for an immediate packet exchange with the controller, and instead returns the result of the most recent identical query which was made by the automated communication feature.

Command type functions are executed much less frequently than query functions. Most of the time the higher layer is monitoring the hardware waiting for a specific hardware state to occur, waiting for a timed delay to expire, or waiting for operator input. Thus command functions represent an interrupt to the normal automated communications.

When a command function is called, its parameter list is copied into temporary storage and is queued for later processing. The next time the Controller Manager services its automated communication feature, it will check the command queue and issue the command to the Communications Manager as a command packet. Multiple commands may have been queued and all commands are issued before another automatic query is issued.

3.3.3 Redundant Safety Enforcement

The Controller Manager enforces the same set of safety rules which each controller enforces. The rules differ and are customized for each type of controller.

The Controller Manager validates all of the command function input parameters it receives against the safety rules before queuing the command. For example, if the higher layer calls a command function to set the centrifuge speed to a value higher than the maximum speed limit, the function will return an error code to the higher layer and the command will not be queued. If the command were queued anyway, the controller would reject the centrifuge speed command for the same reason, and return an error code in the status packet.

The Controller Manager validates all of the received hardware status, queries and command results against the safety rules. For example, if the centrifuge pressure reading is above the maximum pressure limit, the error will be reported to the Fault Manager, which will halt all ongoing machine operations. Under normal circumstances, the controller would have already detected this error and signaled a fault to the hardware Fault Hub.

3.4 Device Manager

The Device Manager builds on the Controller Manager to provide safety rules which involve the status of multiple controllers. It also provides coordination between the controllers. This could include finite state machines to implement a staged series of controller actions and safety rule checks, initiated by one Device Manager API function call.

The Device Manager's lower layer interface is the Controller Manager. Its upper layer interface is a function call API and is shared by the Protocol Manager and the Machine Manager.

The Controller Manager is an intelligent autonomous shell built onto the Communication Manager. It understands unique features of each type of controller and enforces the safety rules for each controller accordingly. If the Controller Manager's owner locked up or otherwise stopped making functions calls into the Controller Manager, it would seem that the safety rules offer enough intelligence to halt all machine operations in the case of a fault. But these rules cannot detect all faults.

What the Controller Manager lacks is any coordinated interaction between the individual controllers, and this could lead to an otherwise preventable problem. An example illustrates this point.

Imagine that the fluid management hardware is implemented so that peristaltic pump control is on one controller and fluid valve control is on another controller. Common sense requires a valve on the pump input to be connected to a fluid source and a valve on the pump output to be connected to a fluid container or an outlet, before the pump is turned.

The Controller Manager's pump controller safety rules would include a maximum speed limit and a velocity error limit to detect an overloaded or seized pump. The Controller Manager's valve controller safety rules would allow a short delay between a valve state change request and confirmation of the valve state change.

Imagine further that the pump input valve is open to a fluid source, the pump output valve is shut, and the pump is then commanded to spin. The pump would turn and build up pressure on the output until a fluid pathway component ruptured, which could cause blood to squirt out of the machine and into an operator's eye.

The Controller Manager's simple rules would not prevent this. If the pump velocity error limit was exceeded before the failure occurred, maybe the pump could be stopped in time to prevent the rupture. If the output valve opened involuntarily due to the pressure, this would violate a safely rule, but only after a problem occurred.

The Device Manager in this case could have an API command function for setting the pump speed. The Device Manager safety rule would return an error if the valves were not set up correctly. The Device Manager would first query the Controller Manager to determine the valve state, then if the valve state was OK it would issue the pump command to the Controller Manager. This is a simple example of multi-controller coordination.

Alternatively, the Device Manager could have an API command function which would initiate a fluid dispense sequence, and a companion status query function to indicate whether the dispense was complete. The dispense command function would have parameters for input and output valve setup, pump speed, and dispense volume. This function could be implemented as a finite state machine with the following steps.

Verify that the function parameter's valve setup meets the safety rules. Query the Controller Manager to obtain the current setup of the valves and the pump speed. Verify that the pump speed is zero. If the valves are not set up correctly, execute the appropriate Controller Manager command functions to correct the setup. Verify the setup again. Execute the appropriate Controller Manager function to spin the pump the correct number of turns. Query the Controller Manager to determine if the pump has stopped, and continue doing this until it has. Signal the Fault Manager if the pump stopped at the wrong position. Execute the appropriate Controller Manager command functions to shut the input and output valves. Verify the change. Set a Device Manager status flag to indicate that the dispense operation is complete.

The Controller Manager requires ownership for access to its control functions. The Device Manager needs access to these to implement some of its control functions. Instead of vying for ownership of the Controller Manager, the Device Manager acts as a proxy for the Protocol Manager or the Machine Manager. So when the Protocol Manager calls a Device Manager control function, the Device Manager acts as the Protocol Manager when it calls a Controller Manager control function.

An implementation issue arises. If a Controller Manager control function should only be called by the Device Manager, the Controller Manager should make the Protocol Manager and the Machine Manager unable to access the control function.

Using the previous examples, the Controller Manager's pump control function should not be accessible to the Protocol Manager and the Machine Manager for two reasons, 1) the Controller Manager alone does not implement the full set of safety rules, 2) if the Device Manager is controlling the pump for the Protocol Manager or the Machine Manager, the higher layer Manager should not be allowed to provide conflicting control by directly calling the Controller Manager.

3.5 Protocol Manager

The Protocol Manager provides three services to the Host Software:
1) automated control of the machine hardware
2) operator control of the automation
3) recovery from a limited duration power outage

The lower layer interfaces of the Protocol Manager are the Device Manager and the Controller Manager, which provide machine control. The higher layer interface is the Machine Manager which provides operator interaction.

3.5.1 Automated Machine Control

The Protocol Manager provides automated control of the machine hardware by processing instructions from a protocol file which is stored on the hard drive.

The protocol instructions represent a batch operation which has a beginning and an end. The instructions are processed in sequential order from beginning to end, with each instruction completed successfully before moving to the next. Any unexpected conditions or errors encountered during protocol instruction processing result in a protocol abort.

A protocol abort causes the Protocol Manager to stop processing instructions and skip to its recovery sequence. Under normal conditions, an abort will not occur and the Protocol Manager will run the recovery sequence immediately after processing the final protocol instruction.

The recovery sequence performs the machine operations which are needed to empty the disposable set and make the machine safe for the operator to manipulate. This allows the operator to remove the disposable set and prepare for a new protocol. The recovery sequence operations include emptying wet set disposable containers to waste, emptying the centrifuge processing bags to waste, and depressurizing the centrifuge.

There are two types of protocol instructions; automation pause, and machine control. Some instructions are accompanied by parameters which specify attributes such as fluid volume, source fluid selection, or destination fluid pathway.

The automation pause instruction provides the basis for machine-requested operator interaction, by causing the Protocol Manager to wait for the operator to assert a resume command. Once the resume command is given, the Protocol Manager continues processing the remaining protocol instructions. The pause instruction is accompanied by an integer parameter which can be used to specify that a certain type of user interaction is required.

For example, the integer parameter value 3 could be used to mean ‘operator must scan barcode for bag 1 now’. The Console Manager which controls the touchscreen would query the Machine Manager and receive status indicating that the Protocol Manager is paused at location 3. The Console Manager could then display a message to alert the operator and then begin monitoring the barcode reader. After processing the barcode information the Console Manager would send the protocol resume command to the Machine Manager. The Machine Manager would forward the protocol resume command to the Protocol Manager.

The machine control instructions are divided into two classes; hardware control, and timing control. Some machine control instructions are implemented in matched pairs to allow multiple operations to execute in parallel.

Hardware control instructions map directly to the functions made available by the Device Manager and the Controller Manager. For example, a ‘valve open saline’ instruction would be implemented by the Protocol Manager as a call to the Controller Manager's valve open function, with saline as a parameter. Note that the Protocol Manager's instructions are not necessarily stored in the protocol file as text and may be stored in a binary format.

There are two types of timing control instructions; delay, and abort. Each is implemented as a countdown timer which expires when the time remaining is reduced to zero. The difference is in what happens when the timer expires.

A delay timer allows the next protocol instruction to be processed when the timer expires. An abort timer aborts the protocol when the timer expires. Normally an abort timer stop instruction will be processed before the abort timer expires, and the abort timer expiration would only occur if something unexpected occurred.

All protocol instructions are sequential because each one is executed fully before the next one is executed. This could lead to a performance problem which significantly extends the time it takes to run a protocol. Some machine control operations will take a relatively long time to complete, like ramping the centrifuge speed, dispensing large volumes to the centrifuge, or pressurizing the centrifuge. If these operations were each implemented by a single protocol instruction; they would each need to be done in series even if there was a need to do them simultaneously.

Thus arises the need for some operations to be implemented as matched pairs of instructions. Each matched pair has a start instruction. Each matched pair also has either a stop instruction or a wait instruction. So the possible matched pairs are start-stop, or start-wait. These instructions are implemented so that multiple start instructions can be executed before any of the stop or wait instructions are executed.

As an example, a start-stop instruction pair could control the air compressor power, or control an abort timer. An example start-wait instruction pair could start the centrifuge and later let the protocol wait until it reached full speed. In between, the protocol could execute any number of other instructions.

3.5.2 Operator Control

The Protocol Manager provides an interface to the Machine Manager which can be used by the Console Manager to allow operator control of the automation. The interface provides a limited set of control operations; protocol file selection, start, pause, resume, and abort. It also provides a limited set of status query operations; instruction processing state, current instruction and parameters, time since protocol start, estimated time remaining to protocol finish.

The protocol file selection interface allows a list of available protocol file names to be generated. Note that the files are already on the hard disk and the Protocol Manager has no capability to add or remove protocol files. The file list can be displayed on the touchscreen and this would allow one of them to be selected by the operator. After a protocol is selected the interface allows the operator to start the protocol.

At any time after the protocol is started and before it is finished, the interface allows the operator to pause or abort the protocol instruction processing. A pause is recognized after the current instruction finishes executing, and will prevent the Protocol Manager from executing the next instruction until the interface's resume command is given. An abort does not wait for the current instruction to complete. It immediately causes the Protocol Manager to skip to its recovery sequence. The recovery sequence cannot be paused or aborted.

When the Protocol Manager executes an automation pause instruction, the interface allows the operator to resume instruction processing.

The Protocol Manager's instruction processor is implemented as a finite state machine. This means that the instruction processor operates in a set of well-defined states, and transitions between states occur in response to instruction processing events and operator interface events. The instruction processor state is represented as integer value.

The Protocol Manager interface provides access to instruction processor's state integer in response to a status query. This value is primarily used to determine whether the instruction processor is started, running, paused, aborting, in the recovery sequence, or finished.

The interface also provides the processor's current instruction and parameters. In the case of an automation pause instruction, the parameter can be used to guide the operator to provide the required interaction. In general, the current instruction information could be used to provide an animation of the machine operations on the touchscreen.

In the production environment where the machine will be deployed, the operator may be controlling more than one machine. The Protocol Manager interface provides the operator with a time remaining estimate which can be used in realtime to determine which machine will finish next.

3.5.3 Power Outage Recovery

The Protocol Manager maintains files on the hard drive with sufficient information about the protocol instruction processing state and consequent changes to machine hardware state to allow a protocol to be temporarily interrupted by a power outage and then resume after power is restored.

Implementation of temporary power outage recovery is by far the most complicated feature of the Protocol Manager. Keeping track of the relevant information and storing it on the hard drive is relatively easy. Interpreting it correctly after power is restored can be complicated.

The power outage may cause the hard drive file system to corrupt or delete any of the files which were being written to when the power was lost. This causes the need for the Protocol Manager to maintain redundant recovery information in separate files so that file data corruption or file loss can be detected when the Protocol Manager restarts after power is restored.

All of the status information needed to resume a protocol after interrupt is stored as a unit in a single file, along with a CRC value which can be used to detect data corruption. This file is termed a recovery file. The status information includes a time stamp, the protocol file name, location within the protocol file of the currently executing instruction, instruction processing state, the status of all controllable hardware, and the readout of all feedback sensors.

A new recovery file is written to the hard disk frequently. A new file is created every ten seconds after a new instruction begins, to capture ongoing state changes during a lengthy instruction, and also each time a protocol instruction is completed. The Protocol Manager keeps up to three of the most recent recovery files, and deletes the oldest recovery file immediately after a new file is written, if the new file is the third recovery file.

After the power loss, the Protocol Manager opens each of the recovery files and checks the file data against the file CRC to verity whether the file is corrupt. If it is, it is deleted. The newest recovery file is used as a starting point for restoring the hardware state and resuming the protocol.

At first it would seem that recovery involves simply restoring the hardware to the state recorded in the recovery file, restoring the protocol instruction processor to the recorded state, and resuming the protocol instruction processing. However, there are situations which require the Protocol Manager to repeat previously completed instructions, meaning that the protocol would resume from a different instruction from the one in the most recent recovery file.

In this case, it would be desirable to use an older recovery file which records the needed hardware state and the correct instruction state, but this file may have been deleted as an outdated file before the power outage. Instead, the Protocol Manager must roll back the effect of the instructions which must be repeated.

A good example of this problem is the case where the power is lost while the machine is expressing supernatant from the centrifuge processing bags to waste after a centrifugation has separated the blood from the supernatant. The recovery file will indicate that the centrifuge speed is the expression speed, which is too low to quickly again separate the blood from the supernatant. So the Protocol Manager will have to back up to the instruction which began the centrifugation.

A further complication of this example would be if the power were lost after one or more or the centrifuge processing bags had expressed to the point where the fluid management hardware detected the supernatant-blood interface. The newest uncorrupted recovery file might not even indicate that this had occurred. This would mean that the interface detector channels would need to be flushed before the centrifugation.

3.6 Machine Manager

The Machine Manager is the highest layer of the machine management services, and it provides the only machine control interface available to the higher layer components of the Host Software.

The lower layer interface of the Machine Manager is a combination of the Protocol Manager, the Device Manager, and the Controller Manager. The higher layers are unable to access these lower layer interfaces directly and must use the Machine Manager interface instead.

To prevent the higher layer components from providing conflicting commands to the hardware, access to some of the Machine Manager's API functions is granted to only one of the components at a time. To access these functions, a higher layer component must succeed in registering itself with the Machine Manager as the owner. If the registration fails, another component is already the owner. A component can relinquish ownership at any time. The Machine Manager exports the underlying interfaces of the machine management services as follows:

1) unlimited access to the status query interface

2) owner-only access to the control interface

3) control interface is limited to automatic or manual mode

The Machine Manager exports the underlying interfaces of the Protocol Manager, the Device Manager, and the Controller Manager by providing similar API functions which look and act like the underlying interface functions. The Machine Manager versions of the API control functions implement the ownership and mode access rules before allowing access to the underlying control functions.

The Machine Manager provides an API function which lets the owner change the control mode from automatic to manual and back. The control mode determines which of the underlying control functions are accessible. In automatic mode, the Protocol Manager control functions are accessible and the Device Manager and Controller Manager control functions are inaccessible. In manual mode, the Device Manager and Controller Manager control functions are accessible and the Protocol Manager control functions are inaccessible.

Regardless of the control mode, automatic or manual, all of the status query API functions are always accessible.

If a higher layer sets the Machine Manager mode to automatic and starts a protocol, the higher layer cannot change the Machine Manager control mode to manual until the protocol completes normally or is aborted by the higher layer. If the higher layer starts a protocol, then relinquishes ownership of the Machine Manager, and a new owner tries to change the Machine Manager mode to manual before the protocol finishes, this will fail too.

4 External Interfaces

The external interfaces of the Host Software provide operator control and monitoring of the machine, and connect the machine to blood bank databases.

4.1 Console Manager

The Console Manager provides the machine's operator interface in the form of graphics and text on the machine's touchscreen display. The text language is selectable at runtime.

The Console Manager supports three increasing levels of operator; novice, expert, and supervisor.

The novice operator interface is verbose and requires more interaction during the blood processing protocol. The expert operator interface is streamlined to perform only the necessary interactions. These two operator levels are only able to use the machine's automated control mode.

The supervisor operator interface provides the operator with the ability to select the novice or expert interfaces. The supervisor interface adds user management and machine diagnostic interfaces. The machine diagnostic interface implements the machines manual control mode.

An important aspect of the Console Manager's automated control mode is the checks and balances it uses to ensure the operator correctly connects the source blood bags and labels the product blood bags correct.

4.2 Inventory Manager

The Inventory Manager provides the machine's interface to the blood bank database. The Inventory Manager provides the machine with a generic database interface, and converts this in realtime to the custom interface required by each type of blood bank database. The Inventory Manager link with the blood bank database is bidirectional and is a live online connection.

When the Console Manager provides a blood unit identification number, the Inventory Manager can query the blood bank database and return to the Console Manager the blood unit's blood type and infectious disease testing results. If the blood type does not match the blood conversion type of the disposable set, or if the blood is infected, the machine can reject the blood unit.

After the blood processing protocol is complete, the Console Manager interfaces with the Inventory Manager to inform the blood bank database that each of the source blood units has been converted to Enzyme-Converted-O (ECO) type blood. Additional information about the conversion can also be uploaded to the database, such as the disposable set lot numbers, operator identifier, or time of conversion. The database could use the disposable set usage formation to maintain the disposable set inventory and procurement.

4.3 Network Manager

The Network Manager provides the machine with a TCP/IP connection to the local network. The Network Manager provides custom interfaces to the Console Manager and Inventory Manager.

The Network Manager's interface to the Console Manager allows remote monitoring of the machine and its blood processing protocol's status. This allows a remote monitoring station to display the status of all blood processing machines within a blood bank.

The Network Manager's interface to the Inventory Manager alleviates the need for the Inventory Manager to know the network connection details of the blood bank database. The Inventory Manager simply makes requests for the information it needs, such as blood unit information, and the Network Manager provides it. The Network Manager locates the blood bank database computer, and acts as a transparent courier of the information between the machine and the database.

### Example Three

### Fault Hub System

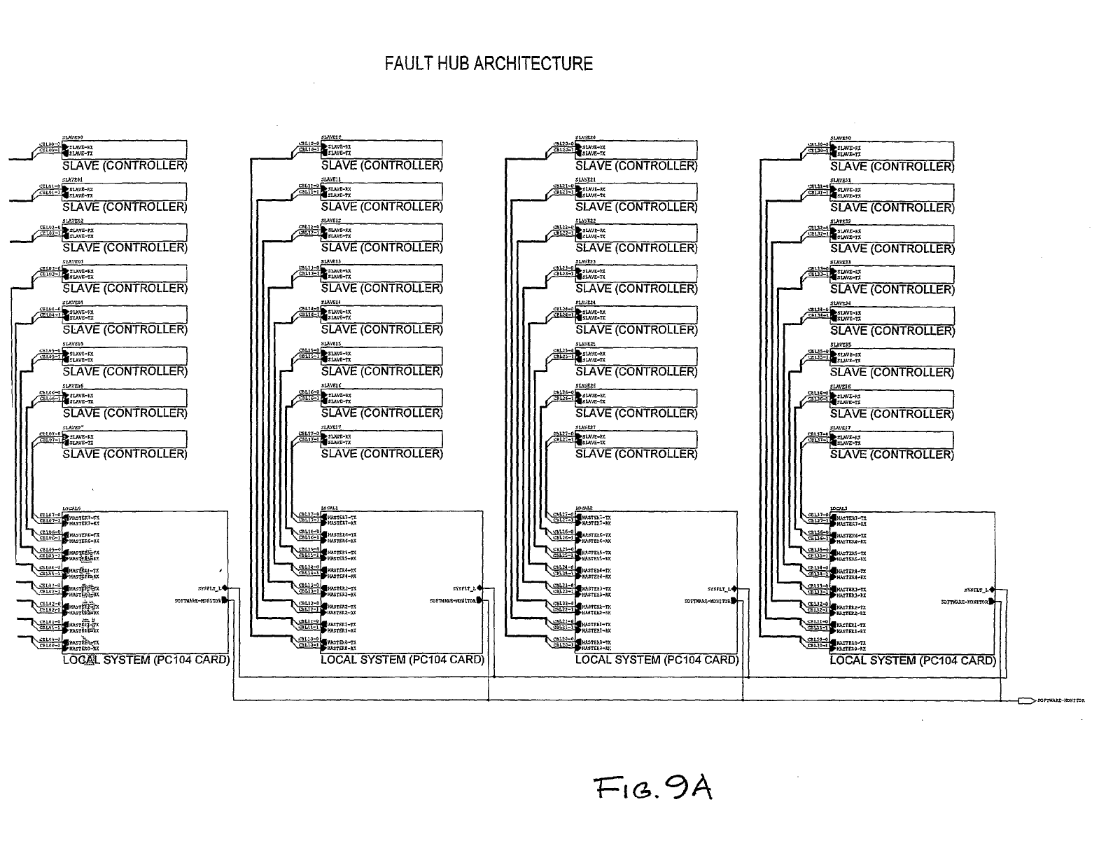

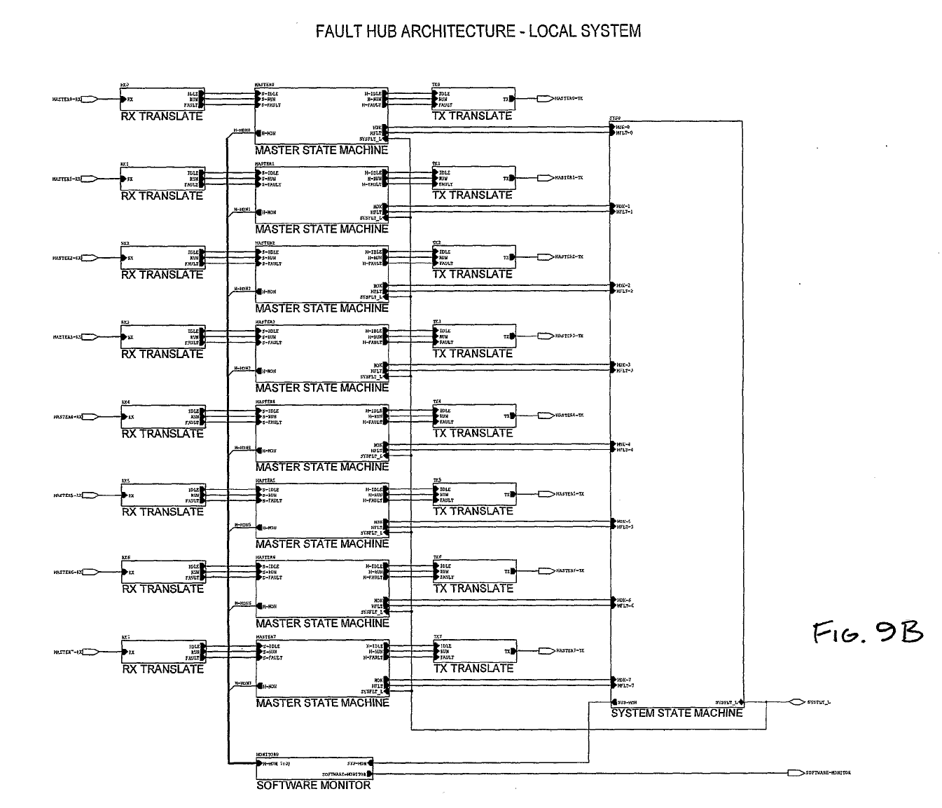

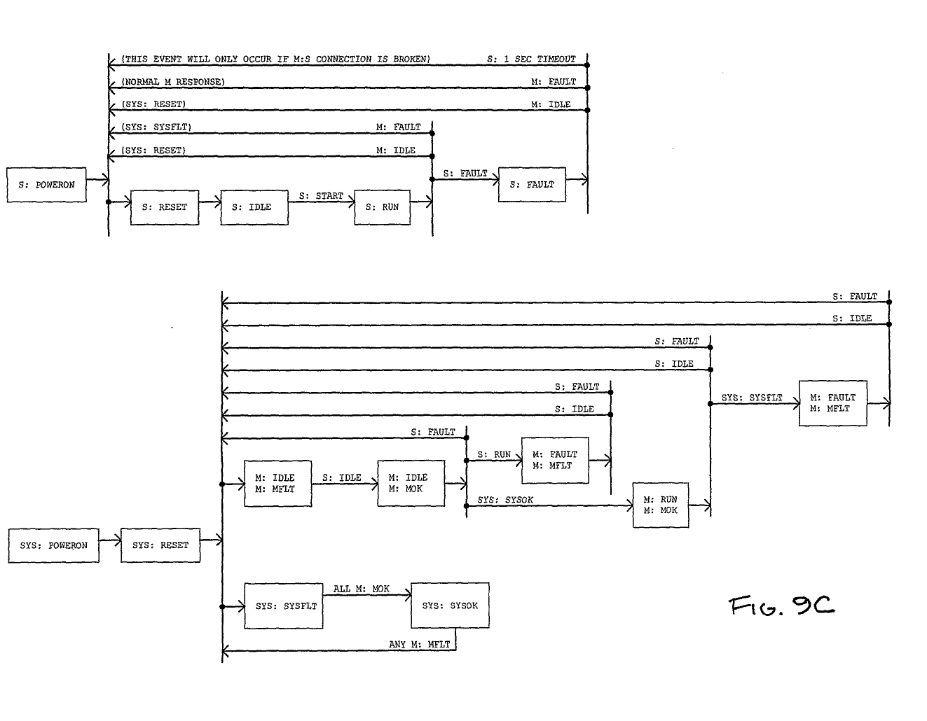

The following describes an example of a fault-hub system and method for use with one or more of the embodiments discussed above. FIGS. 9A, 9B and 9C illustrate the fault hub system.

Overview

The Fault Hub System (see FIGS. 9A-9C) is a collection of Fault Hub Devices to which blood processing controllers are connected. The following terms are defined to generalize the utility of the Fault Hub System specification. The MASTER is a Fault Hub Device. The SLAVE is a ZQ Controller. The SYSTEM is the collection of all MASTERS. Each SLAVE is connected to a dedicated MASTER. Some MASTERS are connected to no SLAVE. All MASTERS are connected to the SYSTEM. Each SLAVE independently indicates to its connected MASTER whether the SLAVE state is IDLE, RUN, or FAULT. Each MASTER indicates to its connected SLAVE the status of the MASTER, which is again IDLE, RUN, or FAULT.

Each MASTER indicates to the SYSTEM whether the MASTER state is MFLT (master fault) or MOK (master OK). The SYSTEM indicates to all connected MASTERS whether the SYSTEM state is SYSFLT (system fault) or SYSOK (system OK). When any MASTER detects a SLAVE state of FAULT, the MASTER indicates MFLT to the SYSTEM. This causes the SYSTEM to indicate SYSFLT to all connected MASTERS. This causes all MASTERS in the SYSTEM to indicate FAULT to their connected SLAVES. So when any SLAVE indicates FAULT to the SYSTEM, the SYSTEM indicates FAULT to all SLAVES. The details of the SYSTEM operation including recovery from the SYSTEM SYSFLT state are described in a manner corresponding to the 7-layer ISO model of open system interconnection.

Physical Layer: Master-Slave

The physical layer signaling between a MASTER and a SLAVE is either RS485 or RS232. This provides one-to-one communications between exactly two transceivers. One transceiver is on the MASTER and other is on the SLAVE. A transceiver consists of a transmitter-receiver pair. A transmitter converts a physical layer bit into an RS485 or RS232 signal level. A receiver detects an RS232 or RS485 signal level and converts it to a physical layer bit. The physical layer bit value is specified as either HI or LO, and its duration is unspecified. When the cable between the transceivers is disconnected, the receiver indicates LO.

Physical Layer: Master-System

The physical layer signaling of the SYSTEM is modeled by an AND logic gate. An AND logic gate has any number of inputs and has only one output. When any input is a LO bit, the output is a LO bit. When all inputs are HI bits, the output is a HI bit. The MFLT and SYSFLT signals are represented by a LO bit. The MOK and SYSOK signals are represented by a HI bit. Each MASTER output is connected to a SYSTEM AND gate input. The SYSTEM AND gate output is the SYSTEM state signal and is connected as an input to all MASTERS. The SYSTEM is realized as collection of separate physical circuits which are connected together by the common SYSTEM state signal. A LOCAL SYSTEM is any one of the physical circuits and is a collection of two or more MASTERS. In a LOCAL SYSTEM circuit, the LOCAL SYSTEM AND logic gate is realized directly by using transistors, discrete logic ICs, or programmable logic ICs. The output of the LOCAL SYSTEM AND gate controls the tri-state enable of a driver which connects directly to the shared SYSTEM state signal. The tri-state driver allows the LOCAL SYSTEM to either pull the SYSTEM state signal to LO and indicate SYSFLT or leave the SYSTEM output unaffected, thereby contributing a SYSOK. When all LOCAL SYSTEMS contribute SYSOK, a pull-up resistor sets the SYSTEM state signal to HI. The durations of the MASTER and SYSTEM signals are unspecified.

Data Link Layer

The data link layer signaling between a MASTER and a SLAVE indicates one of three possible states. They are IDLE, RUN, and FAULT. The RUN state is indicated by a physical layer pulse train. The pulse train is an alternating cycle of HI and LO bits. The alternating cycle is nominally a square wave. The HI and LO bits are each allowed to have a duration no shorter than 1/16 second and no longer than ¼ second. This allows frequency range of 2 Hz to 8 Hz, with a nominal frequency of 4 Hz. The IDLE state is indicated by a LO bit of duration longer than ¼ second. The FAULT state is indicated in one of two ways. One way is HI bit of duration longer than ¼ second. The other is a HI or LO bit of duration shorter than 1/16 second.

Application Layer

The application layer defines the SYSTEM, MASTER, and SLAVE responses to changes in received state values and to asynchronous events. Asynchronous events can occur at any time, independent of the SYSTEM, MASTER, or SLAVE state. The operation of the SYSTEM, MASTER, and SLAVE are described in terms of a finite state machine.

Slave-State Machine

POWERON event: The POWERON event is the starting point of operation and this causes a RESET event.

RESET event: The RESET event causes the SLAVE to indicate IDLE to the MASTER.

IDLE state: The SLAVE waits for an external START event to occur. The MASTER uses an unspecified communication method to cause the START event. While waiting, the MASTER state is ignored and the FAULT event is ignored.

START event: The START event causes the SLAVE to indicate RUN to the MASTER.

RUN state: If the MASTER indicates FAULT, a RESET event is generated. If the MASTER indicates IDLE, a RESET event is generated. The FAULT event causes the SLAVE to indicate FAULT to the MASTER.

FAULT state: If the MASTER indicates FAULT, a RESET event is generated. If the MASTER indicates IDLE, a RESET event is generated. If this state persists for 1 second timeout, a RESET event is generated.

System State Machine

POWERON event: The POWERON event is the starting point of operation and this causes a RESET event for both SYSTEM and MASTER.

RESET event: The RESET event does not directly affect the SYSTEM

state.

MFLT event: When any MASTER indicates MFLT, the SYSTEM indicates SYSFLT.

MOK event: When all MASTERS indicate MOK, the SYSTEM indicates SYSOK.

Master State Machine

RESET event: The RESET event causes the MASTER to indicate IDLE to the SLAVE and MFLT to the SYSTEM.

(IDLE, MFLT) state: The MASTER waits until the SLAVE indicates IDLE, then the MASTER indicates MOK to the SYSTEM.

(IDLE, MOK) state: If the SLAVE indicates FAULT, MASTER indicates IDLE to the SLAVE and MFLT to the SYSTEM. If the SLAVE indicates RUN, MASTER indicated FAULT to the SLAVE and MFLT to the SYSTEM. If the SYSTEM indicates SYSOK, MASTER indicates RUN to the SLAVE and MOK to the SYSTEM.

(FAULT, MFLT) state: The MASTER waits until the SLAVE indicates either IDLE or FAULT, then the MASTER indicates IDLE to the SLAVE and MFLT to the SYSTEM.

### Equivalents

The foregoing description provides for a closed system automated processing device, that has application in many area in the biological, pharmaceutical and industrial arts. Although particular applications for processing of blood are described and referred to throughout, this is not intended to be limiting. A skilled artisan will appreciate that such a device can be adapted to such varying processes, and any such adaptations are intended to be within the scope of the invention. Likewise, the present invention incorporates various mechanical and electrical processes described in our and other patents, patent applications and references. These are cited herein, and are hereby incorporated herein by reference in their entirety.

We claim:

1. A cell processing device comprising:
a. a continuous flow centrifuge, having a structural frame having a plurality of spaced apart structural discs and a plurality of support tubes, the centrifuge further comprising a rotor, the rotor having bearings on each end and a drive system on one end, and the rotor having a hub further comprising a port on one end and a plurality of channels therethrough each channel terminating in an aperture through the hub wall surface;
b. a plurality of chambers disposed along the axis of the rotor, the chambers including substantially flexible processing bags and substantially flexible expressor bags; the processing bags having ports aligning with one or more of the channel apertures on the rotor hub, and the expressor bags having ports aligning with one or more of the channel apertures on the rotor hub;
c. a supply module having a fluid management cassette, the fluid management cassette further comprising a network of pathways and valves, the supply module being further connected to a plurality of fluid and/or air sources, and where the valves are adapted to a system control module;
d. a multi-lumen tube connected at one end to the supply module and at the other end to the hub on the centrifuge rotor, the tube supported by support tube rollers and a rigid arm geared to the centrifuge drive system;
e. a plurality of supply reservoirs in communication with the supply module; and
f. a system control module in electrical communication with the valves, and the centrifuge drive system, the control module having a processor, an instruction set, a fault hub system, and user defined input controls.

2. The device of claim 1, wherein the drive system uses a direct drive motor.

3. The device of claim 1, wherein the centrifuge rotor is adapted a drive pulley and a motor distal to the rotor.

4. The device of claim 1, wherein the valves include protruding valve seats circumscribed by flow-through wells.

5. The device of claim 1, wherein one or more of the supply reservoirs contain a quantity of a glycan modifying enzyme in a buffered solution.

6. The device of claim 1, wherein the glycan modifying enzyme is an alpha-N-acetylgalactosaminidase or an alpha-galactosidase.

7. The device of claim 5, wherein the buffered solution has a pH of 5.0 to 8.0.

8. The device of claim 7, wherein the buffered solution has a pH of 6.0 to 7.8.

9. The device of claim 7, wherein the buffered solution has a pH of 6.5 to 7.5.

10. The device of claim 7, wherein the buffered solution has a pH of 6.8 to 7.5.

11. The device of claim 7, wherein the buffered solution has a pH of 7.0 to 7.3.

12. A method of modifying blood cells comprising: introducing a preparation of isolated blood cells into the processing chambers of the device of claim 1, and reacting said isolated blood cells with a buffered solution of an alpha-N-acetylgalactosaminidase enzyme or an alpha-galactosidase enzyme, or both enzymes, thereby removing immunodominant antigens from the blood cells, separating the enzyme, buffer solution and removed immunodominant antigens from the blood cells by centrifugation and wash steps, and isolating the reacted and washed blood cells.

13. The method of claim 12, wherein the buffered solution has a pH of 5.0 to 8.0.

14. The method of claim 12, wherein the buffered solution has a pH of 6.0 to 7.8.

15. The method of claim 12, wherein the buffered solution has a pH of 6.5 to 7.5.

16. The method of claim 12, wherein the buffered solution has a pH of 6.8 to 7.5.

17. The method of claim 12, wherein the buffered solution has a pH of 7.0 to 7.3.

18. A seroconverted blood cell, wherein the immunodominant antigens are removed by the cell modification method of claim 12.

19. The blood cell of claim 18, wherein the modified blood cell is not a serotype A, B, or AB cell, as determined by standard blood typing methods.

20. A population of packed seroconverted blood cells according to claim 19.

21. A method of treating a subject comprising, identifying a subject in need of a blood transfusion, and providing the subject with a quantity of the seroconverted blood cells of claim 19, thereby restoring blood to the subject and thereby treating the subject.
